SEQUENCE LISTING (254 sequences)

SEQ ID NO: 1 - protein, 24 aa, Ideonella sakaiensis
QTNPYARGPNPTAASLEASAGPFT

SEQ ID NO: 2 - protein, 27 aa, Ideonella sakaiensis
VRSFTVSRPSGYGAGTVYYPTNAGGTV

SEQ ID NO: 3 - protein, 28 aa, Ideonella sakaiensis
GAIAIVPGYTARQSSIKWWGPRLASHGF

SEQ ID NO: 4 - protein, 53 aa, Ideonella sakaiensis
VVITIDTNSTLDQPSSRSSQQMAALRQVASLNGTSSSPIYGKVDTARMGVMGW

SEQ ID NO: 5 - protein, 39 aa, Ideonella sakaiensis
SMGGGGSLISAANNPSLKAAAPQAPWDSSTNFSSVTVPT

SEQ ID NO: 6 - protein, 28 aa, Ideonella sakaiensis
LIFACENDSIAPVNSSALPIYDSMSRNA

SEQ ID NO: 7 - protein, 39 aa, Ideonella sakaiensis
KQFLEINGGSHSCANSGNSNQALIGKKGVAWMKRFMDND

SEQ ID NO: 8 - protein, 25 aa, Ideonella sakaiensis
TRYSTFACENPNSTRVSDFRTANCS

SEQ ID NO: 9 - protein, 23 aa, Thermobifida fusca
ANPYERGPNPTDALLEASSGPFS

SEQ ID NO: 10 - protein, 22 aa, unidentified
SNPYQRGPNPTRSALTADGPFS

SEQ ID NO: 11 - protein, 28 aa, Thermobifida fusca
VSEENVSRLSASGFGGGTIYYPRENNTY

SEQ ID NO: 12 - protein, 29 aa, unidentified
VATYTVSRLSVSGFGGGVIYYPTGTSLTF

SEQ ID NO: 13 - protein, 28 aa, unidentified
GGIAMSPGYTADASSLAWLGRRLASHGF

SEQ ID NO: 14 - protein, 28 aa, Thermobifida fusca
GAVAISPGYTGTEASIAWLGERIASHGF

SEQ ID NO: 15 - protein, 50 aa, Thermobifida fusca
VVITIDTITTLDQPDSRAEQLNAALNHMINRASSTVRSRIDSSRLAVMGH

SEQ ID NO: 16 - protein, 39 aa, Thermobifida fusca
SMGGGGTLRLASQRPDLKAAIPLTPWHLNKNWSSVTVPT

SEQ ID NO: 17 - protein, 38 aa, unidentified
SMGGGGTLRIAEQNPSLKAAVPLTPWHTDKTFNTSVPV

SEQ ID NO: 18 - protein, 29 aa, Thermobifida fusca
LIIGADLDTIAPVATHAKPFYNSLPSSIS

SEQ ID NO: 19 - protein, 36 aa, Thermobifida fusca
KVYVELDNASHFAPNSNNAAISVYTISWMKLWVDND

SEQ ID NO: 20 - protein, 28 aa, Thermobifida fusca
TRYTQFLCPGPRDGLFGEVEEYRSTCPF

SEQ ID NO: 21 - protein, 289 aa, Ideonella sakaiensis
NFPRASRLMQAAVLGGLMAVSAAATAQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 22 - DNA, 783 bp, synthetic construct
  source 1..783: mol_type = other DNA
gccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggcgggatcgccatgtcgccggggtacacggccgacgccagttcgctggcgtggctcgggcggcggttggcatcgcacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 23 - DNA, 780 bp, synthetic construct
  source 1..780: mol_type = other DNA
gccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcttcgcctgcgagaacgacagcatcgccccggtcaactcgtccgccctgccgatctacgacagcatgtcgcgcaatgcgaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 24 - DNA, 777 bp, synthetic construct
  source 1..777: mol_type = other DNA
tccaacccgtaccagcgcggtcccaaccccacgcggagcgcgctcacggccgacgggccgttctcggtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 25 - DNA, 774 bp, synthetic construct
  source 1..774: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacgcgctactccaccttcgcctgcgagaacccgaacagcacccgcgtgtcggacttccgcaccgcgaactgcagc

SEQ ID NO: 26 - DNA, 783 bp, synthetic construct
  source 1..783: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggcgggatcgccatgtcgccggggtacacggccgacgccagttcgctggcgtggctcgggcggcggttggcatcgcacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 27 - DNA, 780 bp, synthetic construct
  source 1..780: mol_type = other DNA
tccaacccgtaccagcgcggtcccaaccccacgcggagcgcgctcacggccgacgggccgttctcggtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcttcgcctgcgagaacgacagcatcgccccggtcaactcgtccgccctgccgatctacgacagcatgtcgcgcaatgcgaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 28 - DNA, 786 bp, synthetic construct
  source 1..786: mol_type = other DNA
cagaccaacccctacgcccgcggcccgaacccgacagccgcctcactcgaagccagcgccggcccgttcaccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 29 - DNA, 786 bp, synthetic construct
  source 1..786: mol_type = other DNA
cagaccaacccctacgcccgcggcccgaacccgacagccgcctcactcgaagccagcgccggcccgttcaccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 30 - DNA, 780 bp, synthetic construct
  source 1..780: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcttcgcctgcgagaacgacagcatcgccccggtcaactcgtccgccctgccgatctacgacagcatgtcgcgcaatgcgaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 31 - DNA, 777 bp, synthetic construct
  source 1..777: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacgcgctactccaccttcgcctgcgagaacccgaacagcacccgcgtgtcggacttccgcaccgcgaactgcagc

SEQ ID NO: 32 - DNA, 780 bp, synthetic construct
  source 1..780: mol_type = other DNA
tccaacccgtaccagcgcggtcccaaccccacgcggagcgcgctcacggccgacgggccgttctcggtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 33 - DNA, 786 bp, synthetic construct
  source 1..786: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggcgggatcgccatgtcgccggggtacacggccgacgccagttcgctggcgtggctcgggcggcggttggcatcgcacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 34 - DNA, 780 bp, synthetic construct
  source 1..780: mol_type = other DNA
tccaacccgtaccagcgcggtcccaaccccacgcggagcgcgctcacggccgacgggccgttctcggtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 35 - DNA, 780 bp, synthetic construct
  source 1..780: mol_type = other DNA
gccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 36 - DNA, 783 bp, synthetic construct
  source 1..783: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcttcgcctgcgagaacgacagcatcgccccggtcaactcgtccgccctgccgatctacgacagcatgtcgcgcaatgcgaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 37 - DNA, 789 bp, synthetic construct
  source 1..789: mol_type = other DNA
cagaccaacccctacgcccgcggcccgaacccgacagccgcctcactcgaagccagcgccggcccgttcaccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 38 - DNA, 783 bp, synthetic construct
  source 1..783: mol_type = other DNA
tccaacccgtaccagcgcggtcccaaccccacgcggagcgcgctcacggccgacgggccgttctcggtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 39 - DNA, 783 bp, synthetic construct
  source 1..783: mol_type = other DNA
gccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 40 - DNA, 783 bp, synthetic construct
  source 1..783: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactcgatgggcggcggcgggaccctccgcatcgcagagcagaacccgtcgctgaaagcggccgtaccgctcacgccgtggcacaccgacaagacgttcaacacgtcggtaccggtgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 41 - DNA, 786 bp, synthetic construct
  source 1..786: mol_type = other DNA
gccaacccctacgagcgcggccccaacccgaccgacgccctgctcgaagccagcagcggccccttctccgtggcaacctacaccgtttcgcggctctcggtgagcggcttcgggggcggggtgatctactaccccacaggcacctcgctgaccttcggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggtgtacgtggagctcgacaatgcgtcgcacttcgcgcccaacagcaacaacgcggcgatctccgtgtacaccatctcgtggatgaagctgtgggtggataacgacacccgctacacccagttcctctgccccggaccgcgcgacggactcttcggcgaggtcgaagagtaccgctccacctgcccgttc

SEQ ID NO: 42 - DNA, 867 bp, Ideonella sakaiensis
  source 1..867: mol_type = genomic DNA
aactttccccgcgcttcccgcctgatgcaggccgccgttctcggcgggctgatggccgtgtcggccgccgccaccgcccagaccaacccctacgcccgcggcccgaacccgacagccgcctcactcgaagccagcgccggcccgttcaccgtgcgctcgttcaccgtgagccgcccgagcggctacggcgccggcaccgtgtactaccccaccaacgccggcggcaccgtgggcgccatcgccatcgtgccgggctacaccgcgcgccagtcgagcatcaaatggtggggcccgcgcctggcctcgcacggcttcgtggtcatcaccatcgacaccaactccacgctcgaccagccgtccagccgctcgtcgcagcagatggccgcgctgcgccaggtggcctcgctcaacggcaccagcagcagcccgatctacggcaaggtcgacaccgcccgcatgggcgtgatgggctggtcgatgggcggtggcggctcgctgatctcggcggccaacaacccgtcgctgaaagccgcggcgccgcaggccccgtgggacagctcgaccaacttctcgtcggtcaccgtgcccacgctgatcttcgcctgcgagaacgacagcatcgccccggtcaactcgtccgccctgccgatctacgacagcatgtcgcgcaatgcgaagcagttcctcgaaatcaacggtggctcgcactcctgcgccaacagcggcaacagcaaccaggcgctgatcggcaagaagggcgtggcctggatgaagcgcttcatggacaacgacacgcgctactccaccttcgcctgcgagaacccgaacagcacccgcgtgtcggacttccgcaccgcgaactgcagc

SEQ ID NO: 43 - DNA, 903 bp, Thermobifida fusca
  source 1..903: mol_type = genomic DNA
atggctgtgatgaccccccgccgggagcgctcttccctgctctcccgagctctgcaagtgacggctgcggctgccacagcgcttgtgaccgcggtcagcctggccgcccccgctcatgccgccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggcctacctggagctggacggcgcaacccacttcgccccgaacatccccaacaagatcatcggcaagtacagcgtcgcctggctcaagcggttcgtcgacaacgacacccgctacacccagttcctctgccccggaccgcgcgacggactcttcggcgaggtcgaagagtaccgctccacctgcccgttc

SEQ ID NO: 44 - DNA, 879 bp, unidentified
  source 1..879: mol_type = genomic DNA
atggacggagttctctggcgagtgcgaaccgcggcgctcatggccgcgctgctcgccctcgcagcctgggcgctggtctgggcttcgcccagcgtcgaggctcaatccaacccgtaccagcgcggtcccaaccccacgcggagcgcgctcacggccgacgggccgttctcggtggcaacctacaccgtttcgcggctctcggtgagcggcttcgggggcggggtgatctactaccccacaggcacctcgctgaccttcggcgggatcgccatgtcgccggggtacacggccgacgccagttcgctggcgtggctcgggcggcggttggcatcgcacggcttcgtggtgctcgtcatcaacacgaactcgcgattcgactaccctgactcccgcgcaagccagctatcggcggcgctgaactacctgcggacgagcagcccctcagccgtacgcgcccggctcgatgcgaaccgcctggccgttgcggggcattcgatgggcggcggcgggaccctccgcatcgcagagcagaacccgtcgctgaaagcggccgtaccgctcacgccgtggcacaccgacaagacgttcaacacgtcggtaccggtgctcatcgtgggagcggaagcggacacggtcgcgcccgtgagccagcacgccattccgttctaccagaacttgccctcgaccacgccgaaggtgtacgtggagctcgacaatgcgtcgcacttcgcgcccaacagcaacaacgcggcgatctccgtgtacaccatctcgtggatgaagctgtgggtggataacgacacccgctaccggcagttcctctgcaacgtgaacgatccggcgctgagcgacttccggacgaacaaccgccactgccag

SEQ ID NO: 45 - DNA, 45 bp, synthetic construct
  source 1..45: mol_type = other DNA
gagatatacatatgcagaccaacccctacgcccgcggcccgaacc

SEQ ID NO: 46 - DNA, 45 bp, synthetic construct
  source 1..45: mol_type = other DNA
gtaggggttggtctgcatatgtatatctccttcttaaagttaaac

SEQ ID NO: 47 - DNA, 45 bp, synthetic construct
  source 1..45: mol_type = other DNA
ggagatatacatatggccaacccctacgagcgcggccccaacccg

SEQ ID NO: 48 - DNA, 45 bp, synthetic construct
  source 1..45: mol_type = other DNA
ctcgtaggggttggccatatgtatatctccttcttaaagttaaac

SEQ ID NO: 49 - DNA, 45 bp, synthetic construct
  source 1..45: mol_type = other DNA
ggagatatacatatggacggagttctctggcgagtgcgaaccgcg

SEQ ID NO: 50 - DNA, 45 bp, synthetic construct
  source 1..45: mol_type = other DNA
ccagagaactccgtccatatgtatatctccttcttaaagttaaac

SEQ ID NO: 51 - DNA, 789 bp, synthetic construct
  source 1..789: mol_type = other DNA
cagaccaacccctacgcccgcggcccgaacccgacagccgcctcactcgaagccagcgccggcccgttcaccgtgcgctcgttcaccgtgagccgcccgagcggctacggcgccggcaccgtgtactaccccaccaacgccggcggcaccgtgggcgccatcgccatcgtgccgggctacaccgcgcgccagtcgagcatcaaatggtggggcccgcgcctggcctcgcacggcttcgtggtcatcaccatcgacaccaactccacgctcgaccagccgtccagccgctcgtcgcagcagatggccgcgctgcgccaggtggcctcgctcaacggcaccagcagcagcccgatctacggcaaggtcgacaccgcccgcatgggcgtgatgggctggtcgatgggcggtggcggctcgctgatctcggcggccaacaacccgtcgctgaaagccgcggcgccgcaggccccgtgggacagctcgaccaacttctcgtcggtcaccgtgcccacgctgatcttcgcctgcgagaacgacagcatcgccccggtcaactcgtccgccctgccgatctacgacagcatgtcgcgcaatgcgaagcagttcctcgaaatcaacggtggctcgcactcctgcgccaacagcggcaacagcaaccaggcgctgatcggcaagaagggcgtggcctggatgaagcgcttcatggacaacgacacgcgctactccaccttcgcctgcgagaacccgaacagcacccgcgtgtcggacttccgcaccgcgaactgcagc

SEQ ID NO: 52 - protein, 263 aa, synthetic construct
QTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 53 - DNA, 783 bp, synthetic construct
  source 1..783: mol_type = other DNA
gccaacccctacgagcgcggccccaacccgaccgacgccctgctcgaagccagcagcggccccttctccgtcagcgaggagaacgtctcccggttgagcgccagcggcttcggcggcggcaccatctactacccgcgggagaacaacacctacggtgcggtggcgatctcccccggctacaccggcactgaggcttccatcgcctggctgggcgagcgcatcgcctcccacggcttcgtcgtcatcaccatcgacaccatcaccaccctcgaccagccggacagccgggcagagcagctcaacgccgcgctgaaccacatgatcaaccgggcgtcctccacggtgcgcagccggatcgatagcagccgactggcggtcatgggccactccatgggcggcggcggcaccctgcgtctggcctcccagcgtcccgacctgaaggccgccatcccgctcaccccgtggcacctcaacaagaactggagcagcgtcaccgtgccgacgctgatcatcggggccgacctcgacacgatcgcgccggtcgccacgcacgcgaaaccgttctacaacagcctgccgagctccatcagcaaggcctacctggagctggacggcgcaacccacttcgccccgaacatccccaacaagatcatcggcaagtacagcgtcgcctggctcaagcggttcgtcgacaacgacacccgctacacccagttcctctgccccggaccgcgcgacggactcttcggcgaggtcgaagagtaccgctccacctgcccgttc

SEQ ID NO: 54 - protein, 261 aa, synthetic construct
ANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 55 - DNA, 774 bp, synthetic construct
  source 1..774: mol_type = other DNA
tccaacccgtaccagcgcggtcccaaccccacgcggagcgcgctcacggccgacgggccgttctcggtggcaacctacaccgtttcgcggctctcggtgagcggcttcgggggcggggtgatctactaccccacaggcacctcgctgaccttcggcgggatcgccatgtcgccggggtacacggccgacgccagttcgctggcgtggctcgggcggcggttggcatcgcacggcttcgtggtgctcgtcatcaacacgaactcgcgattcgactaccctgactcccgcgcaagccagctatcggcggcgctgaactacctgcggacgagcagcccctcagccgtacgcgcccggctcgatgcgaaccgcctggccgttgcggggcattcgatgggcggcggcgggaccctccgcatcgcagagcagaacccgtcgctgaaagcggccgtaccgctcacgccgtggcacaccgacaagacgttcaacacgtcggtaccggtgctcatcgtgggagcggaagcggacacggtcgcgcccgtgagccagcacgccattccgttctaccagaacttgccctcgaccacgccgaaggtgtacgtggagctcgacaatgcgtcgcacttcgcgcccaacagcaacaacgcggcgatctccgtgtacaccatctcgtggatgaagctgtgggtggataacgacacccgctaccggcagttcctctgcaacgtgaacgatccggcgctgagcgacttccggacgaacaaccgccactgccag

SEQ ID NO: 56 - protein, 258 aa, synthetic construct
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 57 - DNA, 45 bp, synthetic construct
  source 1..45: mol_type = other DNA
gaagctgtgggtggataacgacacccgctacacccagttcctctg

SEQ ID NO: 58 - DNA, 35 bp, synthetic construct
  source 1..35: mol_type = other DNA
gctccacgtacaccttgctgatggagctcggcagg

SEQ ID NO: 59 - DNA, 36 bp, synthetic construct
  source 1..36: mol_type = other DNA
ccgagctccatcagcaaggtgtacgtggagctcgac

SEQ ID NO: 60 - DNA, 45 bp, synthetic construct
  source 1..45: mol_type = other DNA
cagaggaactgggtgtagcgggtgtcgttatccacccacagcttc

SEQ ID NO: 61 - DNA, 45 bp, synthetic construct; identical to SEQ ID NO: 57
  source 1..45: mol_type = other DNA

SEQ ID NO: 62 - DNA, 44 bp, synthetic construct
  source 1..44: mol_type = other DNA
ccggcgacatggcgatcccgccgtaggtgttgttctcccgcggg

SEQ ID NO: 63 - DNA, 44 bp, synthetic construct
  source 1..44: mol_type = other DNA
cccgcgggagaacaacacctacggcgggatcgccatgtcgccgg

SEQ ID NO: 64 - DNA, 46 bp, synthetic construct
  source 1..46: mol_type = other DNA
gatggtgtcgatggtgatgacgacgaagccgtgcgatgccaaccgc

SEQ ID NO: 65 - DNA, 45 bp, synthetic construct
  source 1..45: mol_type = other DNA
cggttggcatcgcacggcttcgtcgtcatcaccatcgacaccatc

SEQ ID NO: 66 - DNA, 35 bp, synthetic construct; identical to SEQ ID NO: 58
  source 1..35: mol_type = other DNA

SEQ ID NO: 67 - DNA, 36 bp, synthetic construct; identical to SEQ ID NO: 59
  source 1..36: mol_type = other DNA

SEQ ID NO: 68 - DNA, 45 bp, synthetic construct; identical to SEQ ID NO: 60
  source 1..45: mol_type = other DNA

SEQ ID NO: 69 - DNA, 45 bp, synthetic construct; identical to SEQ ID NO: 57
  source 1..45: mol_type = other DNA

SEQ ID NO: 70 - DNA, 36 bp, synthetic construct
  source 1..36: mol_type = other DNA
cgcaggcgaagatcagcgtcggcacggtgacgctgc

SEQ ID NO: 71 - DNA, 38 bp, synthetic construct
  source 1..38: mol_type = other DNA
cgtcaccgtgccgacgctgatcttcgcctgcgagaacg

SEQ ID NO: 72 - DNA, 36 bp, synthetic construct
  source 1..36: mol_type = other DNA
ctccacgtacaccttcgcattgcgcgacatgctgtc

SEQ ID NO: 73 - DNA, 41 bp, synthetic construct
  source 1..41: mol_type = other DNA
catgtcgcgcaatgcgaaggtgtacgtggagctcgacaatg

SEQ ID NO: 74 - DNA, 45 bp, synthetic construct; identical to SEQ ID NO: 60
  source 1..45: mol_type = other DNA

SEQ ID NO: 75 - DNA, 40 bp, synthetic construct
  source 1..40: mol_type = other DNA
ccacctgcccgttcctcgagcaccaccaccaccaccactg

SEQ ID NO: 76 - DNA, 44 bp, synthetic construct
  source 1..44: mol_type = other DNA
gggagacgttctcctcgctgaccgagaacggcccgtcggccgtg

SEQ ID NO: 77 - DNA, 44 bp, synthetic construct
  source 1..44: mol_type = other DNA
cacggccgacgggccgttctcggtcagcgaggagaacgtctccc

SEQ ID NO: 78 - DNA, 37 bp, synthetic construct
  source 1..37: mol_type = other DNA
gccgccgcccatcgagtggcccatgaccgccagtcgg

SEQ ID NO: 79 - DNA, 35 bp, synthetic construct
  source 1..35: mol_type = other DNA
gcggtcatgggccactcgatgggcggcggcgggac

SEQ ID NO: 80 - DNA, 45 bp, synthetic construct
  source 1..45: mol_type = other DNA
cgaggtcggccccgatgatcagcaccggtaccgacgtgttgaacg

SEQ ID NO: 81 - DNA, 44 bp, synthetic construct
  source 1..44: mol_type = other DNA
gttcaacacgtcggtaccggtgctgatcatcggggccgacctcg

SEQ ID NO: 82 - DNA, 35 bp, synthetic construct; identical to SEQ ID NO: 58
  source 1..35: mol_type = other DNA

SEQ ID NO: 83 - DNA, 36 bp, synthetic construct; identical to SEQ ID NO: 59
  source 1..36: mol_type = other DNA

SEQ ID NO: 84 - DNA, 45 bp, synthetic construct; identical to SEQ ID NO: 60
  source 1..45: mol_type = other DNA

SEQ ID NO: 85 - DNA, 45 bp, synthetic construct; identical to SEQ ID NO: 57
  source 1..45: mol_type = other DNA

SEQ ID NO: 86 - DNA, 34 bp, synthetic construct
  source 1..34: mol_type = other DNA
gtggtggtgctcgaggaacgggcaggtggagcgg

SEQ ID NO: 87 - DNA, 39 bp, synthetic construct
  source 1..39: mol_type = other DNA
cgcgaactgcagcctcgagcaccaccaccaccaccactg

SEQ ID NO: 88 - DNA, 40 bp, synthetic construct
  source 1..40: mol_type = other DNA
ccgcgaaacggtgtaggttgccacggagaaggggccgctg

SEQ ID NO: 89 - DNA, 38 bp, synthetic construct
  source 1..38: mol_type = other DNA
cagcggccccttctccgtggcaacctacaccgtttcgc

SEQ ID NO: 90 - DNA, 40 bp, synthetic construct
  source 1..40: mol_type = other DNA
agatcgccaccgcaccgaaggtcagcgaggtgcctgtggg

SEQ ID NO: 91 - DNA, 40 bp, synthetic construct
  source 1..40: mol_type = other DNA
cacctcgctgaccttcggtgcggtggcgatctcccccggc

SEQ ID NO: 92 - DNA, 37 bp, synthetic construct; identical to SEQ ID NO: 78
  source 1..37: mol_type = other DNA

SEQ ID NO: 93 - DNA, 35 bp, synthetic construct; identical to SEQ ID NO: 79
  source 1..35: mol_type = other DNA

SEQ ID NO: 94 - DNA, 45 bp, synthetic construct; identical to SEQ ID NO: 80
  source 1..45: mol_type = other DNA

SEQ ID NO: 95 - DNA, 44 bp, synthetic construct; identical to SEQ ID NO: 81
  source 1..44: mol_type = other DNA

SEQ ID NO: 96 - DNA, 35 bp, synthetic construct; identical to SEQ ID NO: 58
  source 1..35: mol_type = other DNA

SEQ ID NO: 97 - DNA, 36 bp, synthetic construct; identical to SEQ ID NO: 59
  source 1..36: mol_type = other DNA

SEQ ID NO: 98 - DNA, 37 bp, synthetic construct
  source 1..37: mol_type = other DNA
ggtggagtagcgcgtgtcgttatccacccacagcttc

SEQ ID NO: 99 - DNA, 35 bp, synthetic construct
  source 1..35: mol_type = other DNA
gtgggtggataacgacacgcgctactccaccttcg

SEQ ID NO: 100 - DNA, 45 bp, synthetic construct
  source 1..45: mol_type = other DNA
cagtggtggtggtggtggtgctcgaggctgcagttcgcggtgcgg

SEQ ID NO: 101 - DNA, 45 bp, synthetic construct; identical to SEQ ID NO: 57
  source 1..45: mol_type = other DNA

SEQ ID NO: 102 - DNA, 40 bp, synthetic construct; identical to SEQ ID NO: 88
  source 1..40: mol_type = other DNA

SEQ ID NO: 103 - DNA, 38 bp, synthetic construct; identical to SEQ ID NO: 89
  source 1..38: mol_type = other DNA

SEQ ID NO: 104 - DNA, 46 bp, synthetic construct; identical to SEQ ID NO: 64
  source 1..46: mol_type = other DNA

SEQ ID NO: 105 - DNA, 45 bp, synthetic construct; identical to SEQ ID NO: 65
  source 1..45: mol_type = other DNA

SEQ ID NO: 106 - DNA, 37 bp, synthetic construct; identical to SEQ ID NO: 78
  source 1..37: mol_type = other DNA

SEQ ID NO: 107 - DNA, 35 bp, synthetic construct; identical to SEQ ID NO: 79
  source 1..35: mol_type = other DNA

SEQ ID NO: 108 - DNA, 45 bp, synthetic construct; identical to SEQ ID NO: 80
  source 1..45: mol_type = other DNA

SEQ ID NO: 109 - DNA, 44 bp, synthetic construct; identical to SEQ ID NO: 81
  source 1..44: mol_type = other DNA

SEQ ID NO: 110 - DNA, 35 bp, synthetic construct; identical to SEQ ID NO: 58
  source 1..35: mol_type = other DNA

SEQ ID NO: 111 - DNA, 36 bp, synthetic construct; identical to SEQ ID NO: 59
  source 1..36: mol_type = other DNA

SEQ ID NO: 112 - DNA, 45 bp, synthetic construct; identical to SEQ ID NO: 60
  source 1..45: mol_type = other DNA

SEQ ID NO: 113 - DNA, 40 bp, synthetic construct; identical to SEQ ID NO: 75
  source 1..40: mol_type = other DNA

SEQ ID NO: 114 - DNA, 40 bp, synthetic construct; identical to SEQ ID NO: 90
  source 1..40: mol_type = other DNA

SEQ ID NO: 115 - DNA, 40 bp, synthetic construct; identical to SEQ ID NO: 91
  source 1..40: mol_type = other DNA

SEQ ID NO: 116 - DNA, 36 bp, synthetic construct; identical to SEQ ID NO: 70
  source 1..36: mol_type = other DNA

SEQ ID NO: 117 - DNA, 38 bp, synthetic construct; identical to SEQ ID NO: 71
  source 1..38: mol_type = other DNA

SEQ ID NO: 118 - DNA, 36 bp, synthetic construct; identical to SEQ ID NO: 72
  source 1..36: mol_type = other DNA

SEQ ID NO: 119 - DNA, 41 bp, synthetic construct; identical to SEQ ID NO: 73
  source 1..41: mol_type = other DNA

SEQ ID NO: 120 - DNA, 45 bp, synthetic construct; identical to SEQ ID NO: 60
  source 1..45: mol_type = other DNA

SEQ ID NO: 121 - DNA, 45 bp, synthetic construct; identical to SEQ ID NO: 57
  source 1..45: mol_type = other DNA

SEQ ID NO: 122 - DNA, 34 bp, synthetic construct; identical to SEQ ID NO: 86
  source 1..34: mol_type = other DNA

SEQ ID NO: 123 - DNA, 40 bp, synthetic construct; identical to SEQ ID NO: 75
  source 1..40: mol_type = other DNA

SEQ ID NO: 124 - DNA, 36 bp, synthetic construct
  source 1..36: mol_type = other DNA
cgttctcctcgctgacggtgaacgggccggcgctgg

SEQ ID NO: 125 - DNA, 37 bp, synthetic construct
  source 1..37: mol_type = other DNA
cgccggcccgttcaccgtcagcgaggagaacgtctcc

SEQ ID NO: 126 - DNA, 35 bp, synthetic construct; identical to SEQ ID NO: 58
  source 1..35: mol_type = other DNA

SEQ ID NO: 127 - DNA, 36 bp, synthetic construct; identical to SEQ ID NO: 59
  source 1..36: mol_type = other DNA

SEQ ID NO: 128 - DNA, 45 bp, synthetic construct; identical to SEQ ID NO: 60
  source 1..45: mol_type = other DNA

SEQ ID NO: 129 - DNA, 45 bp, synthetic construct; identical to SEQ ID NO: 57
  source 1..45: mol_type = other DNA

SEQ ID NO: 130 - DNA, 34 bp, synthetic construct; identical to SEQ ID NO: 86
  source 1..34: mol_type = other DNA

SEQ ID NO: 131 - DNA, 40 bp, synthetic construct; identical to SEQ ID NO: 75
  source 1..40: mol_type = other DNA

SEQ ID NO: 132 - DNA, 34 bp, synthetic construct
  source 1..34: mol_type = other DNA
ggtgtaggttgccacggtgaacgggccggcgctg

SEQ ID NO: 133 - DNA, 36 bp, synthetic construct
  source 1..36: mol_type = other DNA
ccggcccgttcaccgtggcaacctacaccgtttcgc

SEQ ID NO: 134 - DNA, 40 bp, synthetic construct; identical to SEQ ID NO: 90
  source 1..40: mol_type = other DNA

SEQ ID NO: 135 - DNA, 40 bp, synthetic construct; identical to SEQ ID NO: 91
  source 1..40: mol_type = other DNA

SEQ ID NO: 136 - DNA, 37 bp, synthetic construct; identical to SEQ ID NO: 78
  source 1..37: mol_type = other DNA

SEQ ID NO: 137 - DNA, 35 bp, synthetic construct; identical to SEQ ID NO: 79
  source 1..35: mol_type = other DNA

SEQ ID NO: 138 - DNA, 45 bp, synthetic construct; identical to SEQ ID NO: 80
  source 1..45: mol_type = other DNA

SEQ ID NO: 139 - DNA, 44 bp, synthetic construct; identical to SEQ ID NO: 81
  source 1..44: mol_type = other DNA

SEQ ID NO: 140 - DNA, 35 bp, synthetic construct; identical to SEQ ID NO: 58
  source 1..35: mol_type = other DNA

SEQ ID NO: 141 - DNA, 36 bp, synthetic construct; identical to SEQ ID NO: 59
  source 1..36: mol_type = other DNA

SEQ ID NO: 142 - DNA, 45 bp, synthetic construct; identical to SEQ ID NO: 60
  source 1..45: mol_type = other DNA

SEQ ID NO: 143 - DNA, 45 bp, synthetic construct; identical to SEQ ID NO: 57
  source 1..45: mol_type = other DNA

SEQ ID NO: 144 - DNA, 34 bp, synthetic construct; identical to SEQ ID NO: 86
  source 1..34: mol_type = other DNA

SEQ ID NO: 145 - DNA, 45 bp, synthetic construct; identical to SEQ ID NO: 57
  source 1..45: mol_type = other DNA

SEQ ID NO: 146 - DNA, 40 bp, synthetic construct; identical to SEQ ID NO: 88
  source 1..40: mol_type = other DNA

SEQ ID NO: 147 - DNA, 38 bp, synthetic construct; identical to SEQ ID NO: 89
  source 1..38: mol_type = other DNA

SEQ ID NO: 148 - DNA, 40 bp, synthetic construct; identical to SEQ ID NO: 90
  source 1..40: mol_type = other DNA

SEQ ID NO: 149 - DNA, 40 bp, synthetic construct; identical to SEQ ID NO: 91
  source 1..40: mol_type = other DNA

SEQ ID NO: 150 - DNA, 37 bp, synthetic construct; identical to SEQ ID NO: 78
  source 1..37: mol_type = other DNA

SEQ ID NO: 151 - DNA, 35 bp, synthetic construct; identical to SEQ ID NO: 79
  source 1..35: mol_type = other DNA

SEQ ID NO: 152 - DNA, 45 bp, synthetic construct
  source 1..45: mol_type = other DNA
cgttctcgcaggcgaagatcagcaccggtaccgacgtgttgaacg

SEQ ID NO: 153 - DNA, 45 bp, synthetic construct
  source 1..45: mol_type = other DNA
cgttcaacacgtcggtaccggtgctgatcttcgcctgcgagaacg

SEQ ID NO: 154 - DNA, 36 bp, synthetic construct; identical to SEQ ID NO: 72
  source 1..36: mol_type = other DNA

SEQ ID NO: 155 - DNA, 41 bp, synthetic construct; identical to SEQ ID NO: 73
  source 1..41: mol_type = other DNA

SEQ ID NO: 156 - DNA, 45 bp, synthetic construct; identical to SEQ ID NO: 60
  source 1..45: mol_type = other DNA

SEQ ID NO: 157 - DNA, 39 bp, synthetic construct; identical to SEQ ID NO: 87
  source 1..39: mol_type = other DNA

SEQ ID NO: 158 - DNA, 40 bp, synthetic construct; identical to SEQ ID NO: 88
  source 1..40: mol_type = other DNA

SEQ ID NO: 159 - DNA, 38 bp, synthetic construct; identical to SEQ ID NO: 89
  source 1..38: mol_type = other DNA

SEQ ID NO: 160 - DNA, 40 bp, synthetic construct; identical to SEQ ID NO: 90
  source 1..40: mol_type = other DNA

SEQ ID NO: 161 - DNA, 40 bp, synthetic construct; identical to SEQ ID NO: 91
  source 1..40: mol_type = other DNA

SEQ ID NO: 162 - DNA, 35 bp, synthetic construct; identical to SEQ ID NO: 58
  source 1..35: mol_type = other DNA

SEQ ID NO: 163 - DNA, 36 bp, synthetic construct; identical to SEQ ID NO: 59
  source 1..36: mol_type = other DNA

SEQ ID NO: 164 - DNA, 37 bp, synthetic construct; identical to SEQ ID NO: 98
  source 1..37: mol_type = other DNA

SEQ ID NO: 165 - DNA, 35 bp, synthetic construct; identical to SEQ ID NO: 99
  source 1..35: mol_type = other DNA

SEQ ID NO: 166 - DNA, 45 bp, synthetic construct; identical to SEQ ID NO: 100
  source 1..45: mol_type = other DNA

SEQ ID NO: 167 - DNA, 40 bp, synthetic construct; identical to SEQ ID NO: 75
  source 1..40: mol_type = other DNA

SEQ ID NO: 168 - DNA, 44 bp, synthetic construct; identical to SEQ ID NO: 76
  source 1..44: mol_type = other DNA

SEQ ID NO: 169 - DNA, 44 bp, synthetic construct; identical to SEQ ID NO: 77
  source 1..44: mol_type = other DNA

SEQ ID NO: 170 - DNA, 35 bp, synthetic construct; identical to SEQ ID NO: 58
  source 1..35: mol_type = other DNA

SEQ ID NO: 171 - DNA, 36 bp, synthetic construct; identical to SEQ ID NO: 59
  source 1..36: mol_type = other DNA

SEQ ID NO: 172 - DNA, 45 bp, synthetic construct; identical to SEQ ID NO: 60
  source 1..45: mol_type = other DNA

SEQ ID NO: 173 - DNA, 45 bp, synthetic construct; identical to SEQ ID NO: 57
  source 1..45: mol_type = other DNA

SEQ ID NO: 174 - DNA, 34 bp, synthetic construct; identical to SEQ ID NO: 86
  source 1..34: mol_type = other DNA

SEQ ID NO: 175 - DNA, 45 bp, synthetic construct; identical to SEQ ID NO: 57
  source 1..45: mol_type = other DNA

SEQ ID NO: 176 - DNA, 40 bp, synthetic construct; identical to SEQ ID NO: 88
  source 1..40: mol_type = other DNA

SEQ ID NO: 177 - DNA, 38 bp, synthetic construct; identical to SEQ ID NO: 89
  source 1..38: mol_type = other DNA

SEQ ID NO: 178 - DNA, 46 bp, synthetic construct; identical to SEQ ID NO: 64
  source 1..46: mol_type = other DNA

SEQ ID NO: 179 - DNA, 45 bp, synthetic construct; identical to SEQ ID NO: 65
  source 1..45: mol_type = other DNA

SEQ ID NO: 180 - DNA, 35 bp, synthetic construct; identical to SEQ ID NO: 58
  source 1..35: mol_type = other DNA

SEQ ID NO: 181 - DNA, 36 bp, synthetic construct; identical to SEQ ID NO: 59
  source 1..36: mol_type = other DNA

SEQ ID NO: 182 - DNA, 45 bp, synthetic construct; identical to SEQ ID NO: 60
  source 1..45: mol_type = other DNA

SEQ ID NO: 183 - DNA, 40 bp, synthetic construct; identical to SEQ ID NO: 75
  source 1..40: mol_type = other DNA

SEQ ID NO: 184 - DNA, 40 bp, synthetic construct; identical to SEQ ID NO: 90
  source 1..40: mol_type = other DNA

SEQ ID NO: 185 - DNA, 40 bp, synthetic construct; identical to SEQ ID NO: 91
  source 1..40: mol_type = other DNA

SEQ ID NO: 186 - DNA, 37 bp, synthetic construct; identical to SEQ ID NO: 78
  source 1..37: mol_type = other DNA

SEQ ID NO: 187 - DNA, 35 bp, synthetic construct; identical to SEQ ID NO: 79
  source 1..35: mol_type = other DNA

SEQ ID NO: 188 - DNA, 45 bp, synthetic construct; identical to SEQ ID NO: 80
  source 1..45: mol_type = other DNA

SEQ ID NO: 189 - DNA, 44 bp, synthetic construct; identical to SEQ ID NO: 81
  source 1..44: mol_type = other DNA

SEQ ID NO: 190 - DNA, 35 bp, synthetic construct; identical to SEQ ID NO: 58
  source 1..35: mol_type = other DNA

SEQ ID NO: 191 - DNA, 36 bp, synthetic construct; identical to SEQ ID NO: 59
  source 1..36: mol_type = other DNA

SEQ ID NO: 192 - DNA, 45 bp, synthetic construct; identical to SEQ ID NO: 60
  source 1..45: mol_type = other DNA

SEQ ID NO: 193 - DNA, 45 bp, synthetic construct; identical to SEQ ID NO: 57
  source 1..45: mol_type = other DNA

SEQ ID NO: 194 - DNA, 34 bp, synthetic construct; identical to SEQ ID NO: 86
  source 1..34: mol_type = other DNA

SEQ ID NO: 195 - DNA, 45 bp, synthetic construct; identical to SEQ ID NO: 57
  source 1..45: mol_type = other DNA

SEQ ID NO: 196 - DNA, 37 bp, synthetic construct; identical to SEQ ID NO: 78
  source 1..37: mol_type = other DNA

SEQ ID NO: 197 - DNA, 35 bp, synthetic construct; identical to SEQ ID NO: 79
  source 1..35: mol_type = other DNA

SEQ ID NO: 198 - DNA, 45 bp, synthetic construct; identical to SEQ ID NO: 80
  source 1..45: mol_type = other DNA

SEQ ID NO: 199 - DNA, 44 bp, synthetic construct; identical to SEQ ID NO: 81
  source 1..44: mol_type = other DNA

SEQ ID NO: 200 - DNA, 35 bp, synthetic construct; identical to SEQ ID NO: 58
  source 1..35: mol_type = other DNA

SEQ ID NO: 201 - DNA, 36 bp, synthetic construct; identical to SEQ ID NO: 59
  source 1..36: mol_type = other DNA

SEQ ID NO: 202 - DNA, 45 bp, synthetic construct; identical to SEQ ID NO: 60
  source 1..45: mol_type = other DNA

SEQ ID NO: 203 - DNA, 45 bp, synthetic construct; identical to SEQ ID NO: 57
  source 1..45: mol_type = other DNA

SEQ ID NO: 204 - DNA, 40 bp, synthetic construct; identical to SEQ ID NO: 88
  source 1..40: mol_type = other DNA

SEQ ID NO: 205 - DNA, 38 bp, synthetic construct; identical to SEQ ID NO: 89
  source 1..38: mol_type = other DNA

SEQ ID NO: 206 - DNA, 40 bp, synthetic construct; identical to SEQ ID NO: 90
  source 1..40: mol_type = other DNA

SEQ ID NO: 207 - DNA, 40 bp, synthetic construct; identical to SEQ ID NO: 91
  source 1..40: mol_type = other DNA

SEQ ID NO: 208 - DNA, 36 bp, synthetic construct; identical to SEQ ID NO: 70
  source 1..36: mol_type = other DNA

SEQ ID NO: 209 - DNA, 38 bp, synthetic construct; identical to SEQ ID NO: 71
  source 1..38: mol_type = other DNA

SEQ ID NO: 210 - DNA, 36 bp, synthetic construct; identical to SEQ ID NO: 72
  source 1..36: mol_type = other DNA

SEQ ID NO: 211 - DNA, 41 bp, synthetic construct; identical to SEQ ID NO: 73
  source 1..41: mol_type = other DNA

SEQ ID NO: 212 - DNA, 45 bp, synthetic construct; identical to SEQ ID NO: 60
  source 1..45: mol_type = other DNA

SEQ ID NO: 213 - DNA, 40 bp, synthetic construct; identical to SEQ ID NO: 75
  source 1..40: mol_type = other DNA

SEQ ID NO: 214 - DNA, 34 bp, synthetic construct; identical to SEQ ID NO: 132
  source 1..34: mol_type = other DNA

SEQ ID NO: 215 - DNA, 36 bp, synthetic construct; identical to SEQ ID NO: 133
  source 1..36: mol_type = other DNA

SEQ ID NO: 216 - DNA, 40 bp, synthetic construct; identical to SEQ ID NO: 90
  source 1..40: mol_type = other DNA

SEQ ID NO: 217 - DNA, 40 bp, synthetic construct; identical to SEQ ID NO: 91
  source 1..40: mol_type = other DNA

SEQ ID NO: 218 - DNA, 35 bp, synthetic construct; identical to SEQ ID NO: 58
  source 1..35: mol_type = other DNA

SEQ ID NO: 219 - DNA, 36 bp, synthetic construct; identical to SEQ ID NO: 59
  source 1..36: mol_type = other DNA

SEQ ID NO: 220 - DNA, 45 bp, synthetic construct; identical to SEQ ID NO: 60
  source 1..45: mol_type = other DNA

SEQ ID NO: 221 - DNA, 45 bp, synthetic construct; identical to SEQ ID NO: 57
  source 1..45: mol_type = other DNA

SEQ ID NO: 222 - DNA, 34 bp, synthetic construct; identical to SEQ ID NO: 86
  source 1..34: mol_type = other DNA

SEQ ID NO: 223 - DNA, 40 bp, synthetic construct; identical to SEQ ID NO: 75
  source 1..40: mol_type = other DNA

SEQ ID NO: 224 - DNA, 40 bp, synthetic construct; identical to SEQ ID NO: 90
  source 1..40: mol_type = other DNA

SEQ ID NO: 225 - DNA, 40 bp, synthetic construct; identical to SEQ ID NO: 91
  source 1..40: mol_type = other DNA

SEQ ID NO: 226 - DNA, 35 bp, synthetic construct; identical to SEQ ID NO: 58
  source 1..35: mol_type = other DNA

SEQ ID NO: 227 - DNA, 36 bp, synthetic construct; identical to SEQ ID NO: 59
  source 1..36: mol_type = other DNA

SEQ ID NO: 228 - DNA, 45 bp, synthetic construct; identical to SEQ ID NO: 60
  source 1..45: mol_type = other DNA

SEQ ID NO: 229 - DNA, 45 bp, synthetic construct; identical to SEQ ID NO: 57
  source 1..45: mol_type = other DNA

SEQ ID NO: 230 - DNA, 34 bp, synthetic construct; identical to SEQ ID NO: 86
  source 1..34: mol_type = other DNA

SEQ ID NO: 231 - DNA, 45 bp, synthetic construct; identical to SEQ ID NO: 57
  source 1..45: mol_type = other DNA

SEQ ID NO: 232 - DNA, 35 bp, synthetic construct; identical to SEQ ID NO: 58
  source 1..35: mol_type = other DNA

SEQ ID NO: 233 - DNA, 36 bp, synthetic construct; identical to SEQ ID NO: 59
  source 1..36: mol_type = other DNA

SEQ ID NO: 234 - DNA, 45 bp, synthetic construct; identical to SEQ ID NO: 60
  source 1..45: mol_type = other DNA

SEQ ID NO: 235 - DNA, 45 bp, synthetic construct; identical to SEQ ID NO: 57
  source 1..45: mol_type = other DNA

SEQ ID NO: 236 - DNA, 40 bp, synthetic construct; identical to SEQ ID NO: 88
  source 1..40: mol_type = other DNA

SEQ ID NO: 237 - DNA, 38 bp, synthetic construct; identical to SEQ ID NO: 89
  source 1..38: mol_type = other DNA

SEQ ID NO: 238 - DNA, 40 bp, synthetic construct; identical to SEQ ID NO: 90
  source 1..40: mol_type = other DNA

SEQ ID NO: 239 - DNA, 40 bp, synthetic construct; identical to SEQ ID NO: 91
  source 1..40: mol_type = other DNA

SEQ ID NO: 240 - DNA, 37 bp, synthetic construct; identical to SEQ ID NO: 78
  source 1..37: mol_type = other DNA

SEQ ID NO: 241 - DNA, 35 bp, synthetic construct; identical to SEQ ID NO: 79
  source 1..35: mol_type = other DNA

SEQ ID NO: 242 - DNA, 45 bp, synthetic construct; identical to SEQ ID NO: 80
  source 1..45: mol_type = other DNA

SEQ ID NO: 243 - DNA, 44 bp, synthetic construct; identical to SEQ ID NO: 81
  source 1..44: mol_type = other DNA

SEQ ID NO: 244 - DNA, 35 bp, synthetic construct; identical to SEQ ID NO: 58
  source 1..35: mol_type = other DNA

SEQ ID NO: 245 - DNA, 36 bp, synthetic construct; identical to SEQ ID NO: 59
  source 1..36: mol_type = other DNA

SEQ ID NO: 246 - DNA, 45 bp, synthetic construct; identical to SEQ ID NO: 60
  source 1..45: mol_type = other DNA

SEQ ID NO: 247 - DNA, 45 bp, synthetic construct; identical to SEQ ID NO: 57
  source 1..45: mol_type = other DNA

SEQ ID NO: 248 - DNA, 40 bp, synthetic construct; identical to SEQ ID NO: 88
  source 1..40: mol_type = other DNA

SEQ ID NO: 249 - DNA, 38 bp, synthetic construct; identical to SEQ ID NO: 89
  source 1..38: mol_type = other DNA

SEQ ID NO: 250 - DNA, 40 bp, synthetic construct; identical to SEQ ID NO: 90
  source 1..40: mol_type = other DNA

SEQ ID NO: 251 - DNA, 40 bp, synthetic construct; identical to SEQ ID NO: 91
  source 1..40: mol_type = other DNA

SEQ ID NO: 252 - DNA, 35 bp, synthetic construct; identical to SEQ ID NO: 58
  source 1..35: mol_type = other DNA

SEQ ID NO: 253 - DNA, 36 bp, synthetic construct; identical to SEQ ID NO: 59
  source 1..36: mol_type = other DNA

SEQ ID NO: 254 - DNA, 45 bp, synthetic construct; identical to SEQ ID NO: 60
  source 1..45: mol_type = other DNA

(19) United States
(12) Patent Application Publication
(10) Pub. No.: US 2025/0154478 A1
(43) Pub. Date: May 15, 2025

(54) ISPETASE-8xCHIMERA RECOMBINASE, ENCODING GENE, RECOMBINANT PLASMID, ENGINEERED STRAIN AND APPLICATIONS

(71) Applicant: BEIJING MEIHAO BIOTECHNOLOGY CO., LTD., Beijing (CN)

(72) Inventors: Wei QI, Beijing (CN); Shengping YOU, Beijing (CN); Mengfan WANG, Beijing (CN); Jiaxing ZHANG, Beijing (CN); Wenhui SHI, Beijing (CN); Qingdian YIN, Beijing (CN); Rongxin SU, Beijing (CN)

(73) Assignee: BEIJING MEIHAO BIOTECHNOLOGY CO., LTD., Beijing (CN)

(21) Appl. No.: 19/028,090

(22) Filed: Jan. 17, 2025

Related U.S. Application Data
(63) Continuation of application No. PCT/CN2024/078000, filed on Feb. 22, 2024.

(30) Foreign Application Priority Data
Sep. 28, 2023 (CN) 202311275023.9

(51) Int. Cl. C12N 9/18; C08J 11/10; C08J 11/24

(52) U.S. Cl. CPC C12N 9/18; C08J 11/105; C08J 11/24; C12Y 301/01074; C08J 2367/02

(57) ABSTRACT

An IsPETase-8×Chimera recombinase includes an amino acid sequence modified from an amino acid sequence of a wild-type PET hydrolase by replacing at least one of the amino acid sequences set forth in SEQ ID NOs: 1 to 8; the amino acid sequence of the wild-type PET hydrolase comprises the amino acid sequences set forth in SEQ ID NOs: 1 to 8.

### CROSS-REFERENCE TO RELATED APPLICATIONS

This application is a continuation of International Application No. PCT/CN2024/078000, filed on Feb. 22, 2024 and entitled “IsPETase-8×Chimera recombinase, encoding gene, recombinant plasmid, engineered strain and applications”, which claims priority to Chinese Patent Application No. 202311275023.9, filed on Sep. 28, 2023 and entitled “IsPETase-8×Chimera recombinase, encoding gene, recombinant plasmid, engineered strain and applications”. The disclosures of the aforementioned applications are hereby incorporated by reference in their entireties.

### INCORPORATION-BY-REFERENCE OF SEQUENCE LISTING

The application contains a sequence listing which has been submitted electronically in XML format and is hereby incorporated by reference in its entirety. Said XML copy, created on Jan. 17, 2025, is named 11300_013018-US0_SL.xml and is 250,062 bytes in size.

### TECHNICAL FIELD

The disclosure relates to the technical field of enzyme engineering, in particular to an IsPETase-8×Chimera recombinase, an encoding gene, a recombinant plasmid, an engineered strain and applications.

### BACKGROUND

As the amount of plastic consumption increases, the impact of plastic waste on the environment has attracted widespread global attention. Polyethylene terephthalate (PET) is one of the most demanded polyester plastics. Generally, major methods for processing PET include landfilling, incineration, pyrolysis, and chemical degradation, all of which consume a large amount of energy and may cause secondary pollution. However, PET is formed by ester bonds of terephthalic acid (TPA) and ethylene glycol (EG), so it can be decomposed by hydrolysis of various enzymes, such as lipases and cutinases. If PET is degraded and TPA is recovered, not only can the impact of PET on the environment be reduced, but the recovered TPA can also bring additional social and economic benefits. Thus, complete depolymerization of PET into monomers is a necessary step to reduce environmental pollution caused by PET, and to obtain a high value-added product TPA.

The biological treatment technology is gradually applied to the treatment of waste plastics due to its characteristics such as high efficiency of degradation, low cost and environmental friendliness. Over the past few years, enzyme-based technology has made significant advances in the treatment of PET waste, providing an environmentally friendly and cost-effective new choice. A variety of enzymes capable of hydrolyzing PET have been discovered, such as esterases, lipases, and cutinases, etc. However, the activity of these PET hydrolases at room temperature is very low, so it is not possible to degrade the remaining PET plastic in situ in natural environment. PETase from bacterial Ideonella sakaiensis, also known as IsPETase, is an enzyme for degrading PET. IsPETase can effectively hydrolyze PET into TPA and other products at room temperature. However, thermal stability and activity at high temperature of IsPETase are poor, and the practical application thereof is still limited.

### SUMMARY

The present disclosure provides an IsPETase-8×Chimera recombinase, an encoding gene, a recombinant plasmid, an engineered strain and applications.

### Technical Problems

The present disclosure provides an IsPETase chimera (IsPETase-8×Chimera recombinase), an encoding gene, a recombinant plasmid, an engineered strain and applications. The recombinase has a relatively high activity and thermal stability, which remedies the deficiency of the existing natural PET hydrolases.

### Technical Solutions

The present disclosure provides an IsPETase-8×Chimera recombinase, and the IsPETase-8×Chimera recombinase comprises an amino acid sequence modified from an amino acid sequence of a wild-type PET hydrolase (set forth in SEQ ID NO: 21) by replacing at least one of the amino acid sequences set forth in SEQ ID NOs: 1 to 8; the amino acid sequence of the wild-type PET hydrolase (set forth in SEQ ID NO: 21) comprises the amino acid sequences set forth in SEQ ID NOs: 1 to 8; where
the amino acid sequence set forth in SEQ ID NO: 1 is replaced by the amino acid sequence set forth in SEQ ID NO: 9 or SEQ ID NO: 10;
the amino acid sequence set forth in SEQ ID NO: 2 is replaced by the amino acid sequence set forth in SEQ ID NO: 11 or SEQ ID NO: 12;
the amino acid sequence set forth in SEQ ID NO: 3 is replaced by the amino acid sequence set forth in SEQ ID NO: 13 or SEQ ID NO: 14;
the amino acid sequence set forth in SEQ ID NO: 4 is replaced by the amino acid sequence set forth in SEQ ID NO: 15;
the amino acid sequence set forth in SEQ ID NO: 5 is replaced by the amino acid sequence set forth in SEQ ID NO: 16 or SEQ ID NO: 17;
the amino acid sequence set forth in SEQ ID NO: 6 is replaced by the amino acid sequence set forth in SEQ ID NO: 18;
the amino acid sequence set forth in SEQ ID NO: 7 is replaced by the amino acid sequence set forth in SEQ ID NO: 19; and
the amino acid sequence set forth in SEQ ID NO: 8 is replaced by the amino acid sequence set forth in SEQ ID NO: 20.

It should be noted that the amino acid sequences set forth in SEQ ID No. 1 to 8 are partial sequences of IsPETase (the amino acid sequence is set forth in SEQ ID No. 21). A peptide segment consisting of the first 26 amino acid residues in the sequence of IsPETase is a signal peptide, and in order to prevent the signal peptide from affecting the correct folding of the protein, it needs to be removed in advance. Thus, the signal peptide is not included in the IsPETase-8×Chimera recombinase.

The IsPETase-8×Chimera recombinase constructed in the present application has a high activity and thermal stability, which makes up for the limitations that the natural IsPETase enzyme has a poor thermal stability and a low reaction activity in practical applications. It can be applied to PET hydrolysis catalytic reactions that have important value in the fields of environment and resources.

In some embodiments, the amino acid sequences set forth in SEQ ID NOs: 2, 3, 4, 5 and 7 are replaced.

As a more preferred technical solution, the amino acid sequence set forth in SEQ ID NO: 3 is replaced by the amino acid sequence set forth in SEQ ID NO: 14.

As an example, the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 17, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

As an example, the amino acid sequence set forth in SEQ ID NO: 1 is replaced by the amino acid sequence set forth in SEQ ID NO: 10, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 17, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

As an example, the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

As an example, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

As an example, the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 11, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

The present disclosure also provides a method for hydrolyzing PET using the IsPETase-8×Chimera recombinase. The method comprises: adding PET to a glycine-sodium hydroxide (NaOH) buffer with a pH of 8.0 to 10.0 and a concentration of 40 millimoles per liter (mM) to 60 mM so that a final concentration of PET is 60 milligrams per milliliter (mg/mL) to 100 mg/mL; adding the IsPETase-8×Chimera recombinase to the solution so that a final concentration of the IsPETase-8×Chimera recombinase is 400 nanomoles per liter (nM) to 600 nM; and incubating the mixture for 1 to 3 days at a temperature of 30° C. to 50° C. and a rotation speed of 100 revolutions per minute (rpm) to 300 rpm.

The present disclosure also provides an encoding gene (a nucleic acid) which encodes the IsPETase-8×Chimera recombinase.

The present disclosure also provides a recombinant plasmid which comprises the nucleic acid.

The present disclosure also provides a method for constructing the recombinant plasmid, and the method comprises:
using plasmid pET-22b-IsPETase, plasmid pET-22b-TfCut2 and plasmid pET-22b-LCC as templates, and performing PCR to obtain linear vector fragments corresponding to each template; mixing each of the linear vector fragments corresponding to each template with a ligase to form circular recombinant plasmids; transforming each of the circular recombinant plasmids into a T1 competent cell, and obtaining a recombinant plasmid pET-22b-IsPETase-cSP, a recombinant plasmid pET-22b-TfCut2-cSP and a recombinant plasmid pET-22b-LCC-cSP by ampicillin-containing medium screening and plasmid extraction; a nucleotide sequence of an IsPETase-cSP gene in the recombinant plasmid pET-22b-IsPETase-cSP is set forth in SEQ ID NO: 51, and an amino acid sequence of an IsPETase-cSP enzyme is set forth in SEQ ID NO: 52; a nucleotide sequence of a TfCut2-cSP gene in the recombinant plasmid pET-22b-TfCut2-cSP is set forth in SEQ ID NO: 53, and an amino acid sequence of a TfCut2-cSP enzyme is set forth in SEQ ID NO: 54; a nucleotide sequence of a LCC-cSP gene in the recombinant plasmid pET-22b-LCC-cSP is set forth in SEQ ID NO: 55, and an amino acid sequence of a LCC-cSP enzyme is set forth in SEQ ID NO: 56. And

Using at least two of the recombinant plasmid pET-22b-IsPETase-cSP, the recombinant plasmid pET-22b-TfCut2-cSP and the recombinant plasmid pET-22b-LCC-cSP as templates to perform PCR separately to generate PCR amplified fragments, ligating the PCR amplified fragments to generate a ligation product, transforming the ligation product into a T1 competent cell, screening using the ampicillin-containing medium and performing plasmid extraction to obtain the recombinant plasmid.

The present disclosure also provides an engineered strain which comprises the recombinant plasmid.

The present disclosure also provides a method for constructing an engineered strain, and the method comprises: transforming the recombinant plasmid into a BL21(DE3) competent cell, and culturing the BL21(DE3) competent cell using an ampicillin-containing medium to obtain a positive recombinant.

Also encompassed by the present disclosure is a method for hydrolyzing PET using the engineered strain. The method comprises: culturing the engineered strain in an LB liquid medium to reach OD600 ranging from 0.7 to 0.9, adding 0.05% to 0.2% of IPTG (isopropylthio-β-galactoside) to the medium, cooling to 14° C. to 20° C. to induce expression for 16 hours (h) to 24 h, centrifuging, and collecting wet cells of the engineered strain;
after breaking down the wet cells of the engineered strain, centrifuging to remove cell debris, eluting the centrifuged supernatant with an elution buffer as an elution solvent by using a Ni-NTA packing column, collecting an eluate, and concentrating the eluate to obtain a protein concentrate containing proteins;
adding the protein concentrate to a glycine-sodium hydroxide buffer with a pH of 8.0 to 10.0 and a concentration of 40 mM to 60 mM, so that the concentration of the proteins is 400 nM to 600 nM, and hydrolyzing PET at 30° C. to 60° C.; wherein a concentration of glycine in the glycine-sodium hydroxide buffer is 50 mM to 200 mM.

### Advantageous Effects of the Disclosure

The IsPETase-8×Chimera recombinase provided in the present disclosure has a relatively high activity and thermal stability. The maximum melting temperature (Tm) can be increased up to 82.71° C. which is 36° C. higher than the Tm of natural IsPETase. The TPA production can reach up to 4856.49 micromoles per liter (μM), which is far higher than that of the wild-type PET hydrolases, such as PET hydrolases IsPETase, PET hydrolases TfCut2 or PET hydrolases LCC at the same temperature. The amino acid fragments of the recombinase respectively come from different native enzyme parents. However, compared with the native enzyme, the recombinase has a new function which the native enzyme does not possess, which is of great significance for achieving efficient degradation of PET. The recombinase can be applied in fields such as plastic waste conversion, resource utilization and environmental protection, and has a wide range of application value.

### DETAILED DESCRIPTION OF ILLUSTRATIVE EMBODIMENTS

In order to better illustrate the present application, further details are given below through specific embodiments and drawings.

The degradation of PET can reduce environmental pollution, and obtain a high value-added product TPA. The enzyme treatment technology can play a role in PET degradation. Currently, a variety of PET hydrolases have been discovered, such as esterases, lipases and cutinases. However, these hydrolases have a low activity at room temperature. IsPETase is capable of effectively hydrolyzing PET at room temperature, but it mainly hydrolyzes amorphous PET. The native IsPETase has a poor stability and activity at high temperature, and its activity will be mostly lost after being incubated at 37° C. for 24 hours, which is not sufficient to maintain a highly effective substrate degradation activity at a higher reaction temperature, seriously limiting its application range.

Based on the study of IsPETase, and combined with rational design and directed evolution, the present application provides an IsPETase-8×Chimera recombinase having high activity and thermal stability by replacing different domains of IsPETase with other amino acid sequences to alter its performance. The recombinase is obtained by replacing at least one of amino acid sequences set forth in SEQ ID NOs: 1 to 8 on the basis of IsPETase (amino acid sequence of IsPETase set forth in SEQ ID NO: 21); where
the amino acid sequence set forth in SEQ ID NO: 1 is replaced by the amino acid sequence set forth in SEQ ID NO: 9 or SEQ ID NO: 10;
the amino acid sequence set forth in SEQ ID NO: 2 is replaced by the amino acid sequence set forth in SEQ ID NO: 11 or SEQ ID NO: 12;
the amino acid sequence set forth in SEQ ID NO: 3 is replaced by the amino acid sequence set forth in SEQ ID NO: 13 or SEQ ID NO: 14;
the amino acid sequence set forth in SEQ ID NO: 4 is replaced by the amino acid sequence set forth in SEQ ID NO: 15;
the amino acid sequence set forth in SEQ ID NO: 5 is replaced by the amino acid sequence set forth in SEQ ID NO: 16 or SEQ ID NO: 17;
the amino acid sequence set forth in SEQ ID NO: 6 is replaced by the amino acid sequence set forth in SEQ ID NO: 18;
the amino acid sequence set forth in SEQ ID NO: 7 is replaced by the amino acid sequence set forth in SEQ ID NO: 19; and/or
the amino acid sequence set forth in SEQ ID NO: 8 is replaced by the amino acid sequence set forth in SEQ ID NO: 20.

The present embodiment also provides a method for constructing the IsPETase-8×Chimera recombinase and an application of the IsPETase-8×Chimera recombinase in the hydrolysis of PET.

The present embodiment also provides a gene (nucleic acid) encoding the IsPETase-8×Chimera recombinase.

The present embodiment also provides a recombinant plasmid which comprises the nucleic acid, a method for constructing the recombinant plasmid, and an engineered strain constructed using the recombinant plasmid.

Also provided in the embodiment of the present application is a method for constructing the engineered strain.

The solution of the present disclosure is illustrated below with reference to specific embodiments.

The reagents in the following examples, if not specified, are either commercially available or obtained according to methods well-known in the art.

### Example 1

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 13, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.22.

### Example 2

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.23.

### Example 3

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 10, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.24.

### Example 4

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, and replacing the amino acid sequence set forth in SEQ ID NO: 7 with the amino acid sequence set forth in SEQ ID NO: 19. The nucleotide sequence encoding the IsPETase-8×Chimera recombinase was set forth in SEQ ID NO.25.

### Example 5

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 13, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.26.

### Example 6

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 10, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.27.

### Example 7

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.28.

### Example 8

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.29.

### Example 9

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.30.

### Example 10

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, and replacing the amino acid sequence set forth in SEQ ID NO: 7 with the amino acid sequence set forth in SEQ ID NO: 19. The nucleotide sequence encoding the IsPETase-8×Chimera recombinase was set forth in SEQ ID NO.31.

### Example 11

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 10, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.32.

### Example 12

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 13, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.33.

### Example 13

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 10, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.34.

### Example 14

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.35.

### Example 15

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.36.

### Example 16

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.37.

### Example 17

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 10, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.38.

### Example 18

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 11, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.39.

### Example 19

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 17, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.40.

### Example 20

The example of the present application provides an IsPETase-8×Chimera recombinase with a high activity and thermal stability. The IsPETase-8×Chimera recombinase was obtained on the basis of IsPETase (amino acid sequence of IsPETase set forth in SEQ ID NO: 21) by replacing the amino acid sequence set forth in SEQ ID NO: 1 with the amino acid sequence set forth in SEQ ID NO: 9, replacing the amino acid sequence set forth in SEQ ID NO: 2 with the amino acid sequence set forth in SEQ ID NO: 12, replacing the amino acid sequence set forth in SEQ ID NO: 3 with the amino acid sequence set forth in SEQ ID NO: 14, replacing the amino acid sequence set forth in SEQ ID NO: 4 with the amino acid sequence set forth in SEQ ID NO: 15, replacing the amino acid sequence set forth in SEQ ID NO: 5 with the amino acid sequence set forth in SEQ ID NO: 16, replacing the amino acid sequence set forth in SEQ ID NO: 6 with the amino acid sequence set forth in SEQ ID NO: 18, replacing the amino acid sequence set forth in SEQ ID NO: 7 with the amino acid sequence set forth in SEQ ID NO: 19, and replacing the amino acid sequence set forth in SEQ ID NO: 8 with the amino acid sequence set forth in SEQ ID NO: 20. The nucleotide sequence encoding the IsPETase-8×Chimera recombinase was set forth in SEQ ID NO.41.

### Example 21

The example of the present application provides an application of the IsPETase-8×Chimera recombinases in Examples 1 to 20 in hydrolyzing PET: a PET film (diameter (p) being 6 millimeters (mm)) was added to a glycine-sodium hydroxide buffer with a pH of 8.0 to 10.0 and a concentration of 50 mM; the IsPETase-8×Chimera recombinase (in Example 1 2, 3, . . . , or 20) was added to the glycine-sodium hydroxide buffer to achieve a final concentration of 400 nM to 600 nM to incubate (hydrolyze) for 1 to 3 days at a temperature of 30° C. to 50° C. and a rotation speed of 100 rpm to 300 rpm.

### Test Example 1

The test example investigated the Tm value of the IsPETase-8×Chimera recombinases in Examples 1 to 20 and the degradation (hydrolyzation) effect on PET films (φ=6 mm).

The Tm value was measured using a Quantitative Real-time Polymerase Chain Reaction (Q-PCR) instrument.

The degradation effect on PET film (φ=6 mm) was performed according to the following method: a PET film (φ=6 mm) was added to a glycine-sodium hydroxide buffer with a pH of 9.0 and a concentration of 50 mM; the IsPETase-8×Chimera recombinase (in Examples 1 to 20) was added to the glycine-sodium hydroxide buffer to achieve a final concentration of 500 nM to incubate (hydrolyze) for 1 day at a temperature of 40° C. to 60° C. and a rotation speed of 250 rpm. The TPA production in the reaction solution obtained after the degradation of PET was detected by High Performance Liquid Chromatography (HPLC). The detection conditions of HPLC were: ultraviolet detector, characteristic absorption peak at 240 nanometers (nm), ZORBAX Eclipse Plus C18 reversed-phase column (filler particle size of 5 m, size of 250 mm×4.6 mm), mobile phase A being a 0.1% formic acid aqueous solution (a ratio of a volume of formic acid to a volume of water being 0.1%), mobile phase B being acetonitrile, the volume ratio of mobile phase B in the total mobile phase rising from 5% to 70% within 20 minutes (min), column temperature being 30° C., injection volume being 10 microliters (L), and flow rate being 0.8 milliliters per minute (mL/min).

The Tm values and TPA production were shown in Table 1.

TABLE 1
The Tm values and TPA production of the
IsPETase-8×Chimera recombinases in Examples 1 to 20

| | | | | | | | | | | Degradation | |
|---|---|---|---|---|---|---|---|---|---|---|---|
| | | | | | | | | | | effect on PET | |
| | | | | | | | | | | TPA | |
| | | | | | | | | | | production | Degradation |
| Examples | SEQ ID NO. | | | | | | | | Tm | (μM) | temperature |
| 1 | 9 | 11 | 13 | 15 | 16 | 18 | 19 | 20 | 78.17° C. | 4095.82 | 50° C. |
| 2 | 9 | 11 | 14 | 15 | 16 | 6 | 19 | 20 | 78.25° C. | 4609.33 | 50° C. |
| 3 | 10 | 11 | 14 | 15 | 17 | 18 | 19 | 20 | 78.29° C. | 4408.21 | 40° C. |
| 4 | 9 | 12 | 14 | 15 | 17 | 18 | 19 | 8 | 78.62° C. | 4105.10 | 50° C. |
| 5 | 9 | 12 | 13 | 15 | 17 | 18 | 19 | 20 | 78.64° C. | 4278.37 | 60° C. |
| 6 | 10 | 12 | 14 | 15 | 16 | 6 | 19 | 20 | 78.76° C. | 4643.21 | 60° C. |
| 7 | 1 | 11 | 14 | 15 | 16 | 18 | 19 | 20 | 78.93° C. | 4216.35 | 40° C. |
| 8 | 1 | 12 | 14 | 15 | 17 | 18 | 19 | 20 | 79.04° C. | 3854.70 | 50° C. |
| 9 | 9 | 12 | 14 | 15 | 17 | 6 | 19 | 20 | 79.19° C. | 3907.70 | 40° C. |
| 10 | 9 | 12 | 14 | 15 | 16 | 18 | 19 | 8 | 79.30° C. | 4453.71 | 60° C. |
| 11 | 10 | 11 | 14 | 15 | 16 | 18 | 19 | 20 | 79.50° C. | 4780.30 | 50° C. |
| 12 | 9 | 12 | 13 | 15 | 16 | 18 | 19 | 20 | 79.66° C. | 4856.49 | 60° C. |
| 13 | 10 | 12 | 14 | 15 | 17 | 18 | 19 | 20 | 79.70° C. | 4679.64 | 60° C. |
| 14 | 9 | 11 | 14 | 15 | 17 | 18 | 19 | 20 | 79.78° C. | 4308.71 | 40° C. |
| 15 | 9 | 12 | 14 | 15 | 16 | 6 | 19 | 20 | 80.12° C. | 4633.74 | 50° C. |
| 16 | 1 | 12 | 14 | 15 | 16 | 18 | 19 | 20 | 80.39° C. | 4570.76 | 50° C. |
| 17 | 10 | 12 | 14 | 15 | 16 | 18 | 19 | 20 | 80.89° C. | 4032.69 | 40° C. |
| 18 | 9 | 11 | 14 | 15 | 16 | 18 | 19 | 20 | 81.23° C. | 4823.61 | 60° C. |
| 19 | 9 | 12 | 14 | 15 | 17 | 18 | 19 | 20 | 81.28° C. | 3747.90 | 40° C. |
| 20 | 9 | 12 | 14 | 15 | 16 | 18 | 19 | 20 | 82.71° C. | 4111.56 | 50° C. |

The present test example also investigated the Tm and TPA production of wild-type PET hydrolases IsPETase, TfCut2 and LCC in the same manner, and the results were as shown in Table 2.

TABLE 2
The Tm and TPA production of wild-type PET hydrolases IsPETase, TfCut2 and LCC

| Wild-type | | | | | | |
|---|---|---|---|---|---|---|
| PET | | TPA production (μM) | | | | |
| hydrolases | Tm | 40° C. | 50° C. | 60° C. | 70° C. | 80° C. |
| IsPETase | 46.7° C. | 216.78 | 32.33 | / | / | / |
| TfCut2 | 72.7° C. | 207.81 | 1266.49 | 3435.03 | 166.94 | 24.03 |
| LCC | 86.6° C. | 1085.35 | 2299.36 | 3701.64 | 2405.92 | 250.05 |

### Example 22

The example of the present application provides a recombinant plasmid containing a gene encoding the PET hydrolase IsPETase-8×Chimera recombinase in Examples 1 to 20 and a construction method therefor, and the construction method comprises:

Step 1, the construction of recombinant plasmid pET-22b-IsPETase-cSP, recombinant plasmid pET-22b-TfCut2-cSP and recombinant plasmid pET-22b-LCC-cSP.

The construction of recombinant plasmid pET-22b-IsPETase: the gene of wild-type PET hydrolase IsPETase (SEQ ID NO: 42) was connected to a pET-22b plasmid to obtain the recombinant plasmid pET-22b-IsPETase.

The construction of recombinant plasmid pET-22b-TfCut2: the gene of wild-type PET hydrolase TfCut2 (SEQ ID NO: 43) was connected to a pET-22b plasmid to obtain the recombinant plasmid pET-22b-TfCut2.

The construction of recombinant plasmid pET-22b-LCC: the gene of wild-type PET hydrolase LCC (SEQ ID NO: 44) was connected to a pET-22b plasmid to obtain the recombinant plasmid pET-22b-LCC.

The gene of wild-type PET hydrolase IsPETase was sourced from Ideonella sakaiensis (GI:1028065175), the gene of wild-type PET hydrolase TfCut2 was sourced from Thermobifida fusca (GenBank: JN129500.1), and the gene of wild-type PET hydrolase LCC was sourced from uncultured bacterium (GenBank: HQ704839.1).

The pET-22b plasmid was commercially available.

A method for preparing the recombinant plasmid (recombinant plasmids pET-22b-IsPETase-cSP, pET-22b-TfCu2-cSP and pET-22b-LCC-cSP) comprises the following specific steps:

(1), some primers were designed using the recombinant plasmids PET-22b-IsPETase, pET-22b-TfCut2 and pET-22b-LCC as templates, respectively, according to the 2×TransStart® FastPfu PCR SuperMix kit of TransGen Biotech. The primers were as following:

IsPETase-cSP-F:
(SEQ ID No. 45)
5′-GAGATATACATATGCAGACCAACCCCTACGCCCGCGGCCCGAACC.
IsPETase-cSP-R:
(SEQ ID No.46)
5′-GTAGGGGTTGGTCTGCATATGTATATCTCCTTCTTAAAGTTAAAC.
TfCut2-cSP-F:
(SEQ ID NO. 47)
5′-GGAGATATAcatatgGCCAACCCCTACGAGCGCGGCCCCAACCCG.
TfCut2-cSP-R:
(SEQ ID No. 48)
5′-CTCGTAGGGGTTGGCcatatgTATATCTCCTTCTTAAAGTTAAAC.
LCC-cSP-F:
(SEQ ID No. 49)
5′-GGAGATATAcatatgGACGGAGTTCTCTGGCGAGTGCGAACCGCG.
LCC-cSP-R:
(SEQ ID No. 50)
5′-CCAGAGAACTCCGTCcatatgTATATCTCCTTCTTAAAGTTAAAC.

(2), PCR was performed to obtain linear vector fragments of 6.2 kilobases (kb), 6.2 kb and 6.2 kb corresponding to each template. Each of the obtained linear vector fragments corresponding to each template was mixed with a ligase to form circular recombinant plasmids (three circular recombinant plasmids were obtained). Each of the circular recombinant plasmids was transformed into a T1 competent cell respectively to obtain a recombinant plasmid pET-22b-IsPETase-cSP, a recombinant plasmid pET-22b-TfCut2-cSP and a recombinant plasmid pET-22b-LCC-cSP by ampicillin-containing medium screening, plasmid extraction and sequencing verification. A nucleotide sequence of an IsPETase-cSP (cut off signal peptide, cSP) gene in the recombinant plasmid pET-22b-IsPETase-cSP was set forth in SEQ ID NO: 51, and an amino acid sequence of an IsPETase-cSP enzyme was set forth in SEQ ID NO: 52; a nucleotide sequence of a TfCut2-cSP gene in the recombinant plasmid pET-22b-TfCut2-cSP was set forth in SEQ ID NO: 53, and an amino acid sequence of a TfCut2-cSP enzyme was set forth in SEQ ID NO: 54; a nucleotide sequence of a LCC-cSP gene in the recombinant plasmid pET-22b-LCC-cSP was set forth in SEQ ID NO: 55, and an amino acid sequence of a LCC-cSP enzyme was set forth in SEQ ID NO: 56.

The formulations of ampicillin-containing medium were: 5 grams per liter (g/L) of yeast extract, 10 g/L of tryptone, 10 g/L of sodium chloride, 15 g/L of agar powder, and 50 milligrams per liter (mg/L) of ampicillin.

The PCR reaction system and PCR reaction program were performed with reference to the requirements of the 2×TransStart® FastPfu PCR SuperMix kit of TransGen Biotech, as shown in Table 3:

TABLE 3
PCR reaction system

| PCR reaction system (50 μL) | | PCR reaction program | |
|---|---|---|---|
| Template | Variable | | 95° C., 2 min |
| Forward primer | 1 μL | Cycle | 95° C., 20 seconds (s) |
| (10 μM) | | number | |
| Reverse primer | 1 μL | being | Tm −5° C., 20 s |
| (10 μM) | | 30 to 35 | |
| 2 × TransStart ® | 25 μL | | 72° C., 4 kb/min |
| FastPfu PCR SuperMix | | | |
| Nuclease-free Water | Variable | | 72° C., 5 min |

Step 2, the construction of a recombinant plasmid containing a gene encoding the IsPETase-8×Chimera recombinase (as exemplified in Example 18).

The primers required for recombination were designed and synthesized.

8xchimera 18-1-F:
(SEQ ID No. 57)
5′-GAAGCTGTGGGTGGATAACGACACCCGCTACACCCAGTTCCTCTG.
8xchimera 18-1-R:
(SEQ ID No. 58)
5′-GCTCCACGTACACCTTGCTGATGGAGCTCGGCAGG.
8xchimera 18-2-F:
(SEQ ID No. 59)
5′-CCGAGCTCCATCAGCAAGGTGTACGTGGAGCTCGAC.
8xchimera 18-2-R:
(SEQ ID NO. 60)
5′-CAGAGGAACTGGGTGTAGCGGGTGTCGTTATCCACCCACAGCTTC.

A recombinant plasmid containing a gene encoding the IsPETase-8×Chimera recombinase in Example 18 was constructed by PCR (the amplification system was the same as that in Table 3) and by seamless ligation as required for a seamless recombination kit. The process mainly comprised three steps: (1) PCR was performed (PCR reaction program: 95° C. for 2 min; 95° C. for 20 s, 76° C. for 20 s, 72° C. for 5 min, 30 cycles; 72° C. for 5 min) using the recombinant plasmid pET-22b-TfCut2-cSP as a template, and using 8×chimera 18-1-F and 8×chimera 18-1-R as a forward (upstream) primer and a reverse (downstream) primer, respectively; DMT enzyme digestion, nucleic acid electrophoresis and degumming recovery were performed on the PCR product to obtain a first group of purified gene fragments; (2) PCR was performed (PCR reaction program: 95° C. for 2 min; 95° C. for 20 s, 76° C. for 20 s, 72° C. for 5 min, 30 cycles; 72° C. for 5 min) using the recombinant plasmid pET-22b-LCC-cSP as a template, 8×chimera 18-2-F and 8×chimera 18-2-R as a forward primer and a reverse primer, respectively; DMT enzyme digestion, nucleic acid electrophoresis and degumming recovery were performed on the PCR product to obtain a second group of purified gene fragments; and (3) the first group of purified gene fragments and the second group of purified gene fragments were ligated with a seamless recombination ligase to transform into a T1 competent cell to obtain the recombinant plasmid containing the gene encoding the IsPETase-8×Chimera recombinase in Example 18 by means of ampicillin-containing medium screening, plasmid extraction and sequencing verification. The nucleotide sequence of a gene encoding the IsPETase-8×Chimera 18 recombinase was set forth in SEQ ID No. 39.

The formulations of ampicillin-containing medium were: 5 g/L of yeast extract, 10 g/L of tryptone, 10 g/L of sodium chloride, 15 g/L of agar powder, and 50 mg/L of ampicillin. The seamless recombination ligase was a seamless cloning enzyme, and 2× MultiF Seamless Assembly Mix produced by ABclonal was used in the present application.

When constructing the IsPETase-8×Chimera recombinases in other examples, the primers required for recombination corresponding to each recombinase (see Table 4) were designed and synthesized, and the recombinant plasmids containing genes encoding the respective recombinases were constructed by seamless ligation and PCR using the recombinant plasmid pET-22b-IsPETase-cSP, the recombinant plasmid pET-22b-TfCut2-cSP and the recombinant plasmid pET-22b-LCC-cSP in step 1 according to the requirements of the seamless recombination kit.

TABLE 4
Primers and templates corresponding to IsPETase-8xChimera recombinases in Examples 1 to 20

| | | Forward primer 1 | Reverse primer 1 |
|---|---|---|---|
| Examples | Template 1 | (SEQ ID No. 61) | (SEQ ID No. 62) |
| 1 | pET-22b- | GAAGCTGTGGGTGGATAACGA | CCGGCGACATGGCGATCCCGCC |
| | TfCut2-cSP | CACCCGCTACACCCAGTTCCTC | GTAGGTGTTGTTCTCCCGCGGG |
| | | TG | |
| | Template 2 | Forward primer 2 (SEQ ID No. 63) | Reverse primer 2 (SEQ ID No. 64) |
| | pET-22b- | CCCGCGGGAGAACAACACCTA | GATGGTGTCGATGGTGATGACG |
| | LCC-cSP | CGGCGGGATCGCCATGTCGCCG | ACGAAGCCGTGCGATGCCAAC |
| | | G | CGC |
| | Template 3 | Forward primer 3 (SEQ ID No. 65) | Reverse primer 3 (SEQ ID No. 66) |
| | pET-22b- | CGGTTGGCATCGCACGGCTTCG | GCTCCACGTACACCTTGCTGAT |
| | TfCut2-cSP | TCGTCATCACCATCGACACCAT | GGAGCTCGGCAGG |
| | | C | |
| | Template 4 | Forward primer 4 (SEQ ID No. 67) | Reverse primer 4 (SEQ ID No. 68) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 2 | Template 1 | Forward primer 1 (SEQ ID No. 69) | Reverse primer 1 (SEQ ID No. 70) |
| | pET-22b- | GAAGCTGTGGGTGGATAACGA | cgcaggcgaagatcagCGTCGGCACGG |
| | TfCut2-cSP | CACCCGCTACACCCAGTTCCTC | TGACGCTGC |
| | | TG | |
| | Template 2 | Forward primer 2 (SEQ ID No. 71) | Reverse primer 2 (SEQ ID No. 72) |
| | pET-22b- | CGTCACCGTGCCGACGctgatcttcg | CTCCACGTACACCTTcgcattgcgcg |
| | IsPETase-cSP | cctgcgagaacg | acatgctgtc |
| | Template 3 | Forward primer 3 (SEQ ID No. 73) | Reverse primer 3 (SEQ ID No. 74) |
| | pET-22b- | catgtcgcgcaatgcgAAGGTGTACGT | CAGAGGAACTGGGTGTAGCGG |
| | LCC-cSP | GGAGCTCGACAATG | GTGTCGTTATCCACCCACAGCT |
| | | | TC |
| 3 | Template 1 | Forward primer 1 (SEQ ID No. 75) | Reverse primer 1 (SEQ ID No. 76) |
| | pET-22b- | CCACCTGCCCGTTCctcgagCACC | GGGAGACGTTCTCCTCGCTGAC |
| | LCC-cSP | ACCACCACCACCACTG | CGAGAACGGCCCGTCGGCCGT |
| | | | G |
| | Template 2 | Forward primer 2 (SEQ ID No. 77) | Reverse primer 2 (SEQ ID No. 78) |
| | pET-22b- | CACGGCCGACGGGCCGTTCTCG | GCCGCCGCCCATCGAGTGGCCC |
| | TfCut2-cSP | GTCAGCGAGGAGAACGTCTCC | ATGACCGCCAGTCGG |
| | | C | |
| | Template 3 | Forward primer 3 (SEQ ID No. 79) | Reverse primer 3 (SEQ ID No. 80) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 4 | Forward primer 4 (SEQ ID No. 81) | Reverse primer 4 (SEQ ID No. 82) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 5 | Forward primer 5 (SEQ ID No. 83) | Reverse primer 5 (SEQ ID No. 84) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 6 | Forward primer 6 (SEQ ID No. 85) | Reverse primer 6 (SEQ ID No. 86) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 4 | Template 1 | Forward primer 1 (SEQ ID No. 87) | Reverse primer 1 (SEQ ID No. 88 |
| | pET-22b- | cgcgaactgcagcctcgagCACCACCAC | CCGCGAAACGGTGTAGGTTGCC |
| | TfCut2-cSP | CACCACCACTG | ACGGAGAAGGGGCCGCTG |
| | Template 2 | Forward primer 2 (SEQ ID No.89) | Reverse primer 2 (SEQ ID No. 90) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 91) | Reverse primer 3 (SEQ ID No. 92) |
| | pET-22b- | CACCTCGCTGACCTTCGGTGCG | GCCGCCGCCCATCGAGTGGCCC |
| | TfCut2-cSP | GTGGCGATCTCCCCCGGC | ATGACCGCCAGTCGG |
| | Template 4 | Forward primer 4 (SEQ ID No. 93) | Reverse primer 4 (SEQ ID No. 94) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 5 | Forward primer 5 (SEQ ID No. 95) | Reverse primer 5 (SEQ ID No. 96) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 6 | Forward primer 6 (SEQ ID No. 97) | Reverse primer 6 (SEQ ID No. 98) |
| | pET-22b- | CCGAGCTCCATCAGCAAGGTGT | ggtggagtagcgcgtGTCGTTATCCAC |
| | LCC-cSP | ACGTGGAGCTCGAC | CCACAGCTTC |
| | Template 7 | Forward primer 7 (SEQ ID No. 99) | Reverse primer 7 (SEQ ID No. 100) |
| | pET-22b- | GTGGGTGGATAACGACacgcgctac | CAGTGGTGGTGGTGGTGGTGctc |
| | IsPETase-cSP | tccaccttcg | gaggctgcagttcgcggtgcgg |
| 5 | Template 1 | Forward primer 1 (SEQ ID No. 101) | Reverse primer 1 (SEQ ID No. 102) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 103) | Reverse primer 2 (SEQ ID No. 104) |
| | pET-22b- | CAGCGGCCCCTTCTCCGTGGCA | GATGGTGTCGATGGTGATGACG |
| | LCC-cSP | ACCTACACCGTTTCGC | ACGAAGCCGTGCGATGCCAAC |
| | | | CGC |
| | Template 3 | Forward primer 3 (SEQ ID No. 105) | Reverse primer 3 (SEQ ID No. 106) |
| | pET-22b- | CGGTTGGCATCGCACGGCTTCG | GCCGCCGCCCATCGAGTGGCCC |
| | TfCut2-cSP | TCGTCATCACCATCGACACCAT | ATGACCGCCAGTCGG |
| | | C | |
| | Template 4 | Forward primer 4 (SEQ ID No. 107) | Reverse primer 4 (SEQ ID No. 108) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 5 | Forward primer 5 (SEQ ID No. 109) | Reverse primer 5 (SEQ ID No. 110) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 6 | Forward primer 6 (SEQ ID No. 111) | Reverse primer 6 (SEQ ID No. 112) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 6 | Template 1 | Forward primer 1 (SEQ ID No. 113) | Reverse primer 1 (SEQ ID No. 114) |
| | pET-22b- | CCACCTGCCCGTTCctcgagCACC | AGATCGCCACCGCACCGAAGG |
| | LCC-cSP | ACCACCACCACCACTG | TCAGCGAGGTGCCTGTGGG |
| | Template 2 | Forward primer 2 (SEQ ID No. 114) | Reverse primer 2 (SEQ ID No. 116) |
| | pET-22b- | CACCTCGCTGACCTTCGGTGCG | cgcaggcgaagatcagCGTCGGCACGG |
| | TfCut2-cSP | GTGGCGATCTCCCCCGGC | TGACGCTGC |
| | Template 3 | Forward primer 3 (SEQ ID No. 117) | Reverse primer 3 (SEQ ID No. 118) |
| | pET-22b- | CGTCACCGTGCCGACGctgatcttcg | CTCCACGTACACCTTcgcattgcgcg |
| | IsPETase-cSP | cctgcgagaacg | acatgctgtc |
| | Template 4 | Forward primer 4 (SEQ ID No. 119) | Reverse primer 4 (SEQ ID No. 120) |
| | pET-22b- | catgtcgcgcaatgcgAAGGTGTACGT | CAGAGGAACTGGGTGTAGCGG |
| | LCC-cSP | GGAGCTCGACAATG | GTGTCGTTATCCACCCACAGCT |
| | | | TC |
| | Template 5 | Forward primer 5 (SEQ ID No. 121) | Reverse primer 5 (SEQ ID No. 122) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 7 | Template 1 | Forward primer 1 (SEQ ID No. 123) | Reverse primer 1 (SEQ ID No. 124) |
| | pET-22b- | CCACCTGCCCGTTCctcgagCACC | CGTTCTCCTCGCTGACggtgaacgg |
| | IsPETase-cSP | ACCACCACCACCACTG | gccggcgctgg |
| | Template 2 | Forward primer 2 (SEQ ID No. 125) | Reverse primer 2 (SEQ ID No. 126) |
| | pET-22b- | cgccggcccgttcaccGTCAGCGAGGA | GCTCCACGTACACCTTGCTGAT |
| | TfCut2-cSP | GAACGTCTCC | GGAGCTCGGCAGG |
| | Template 3 | Forward primer 3 (SEQ ID No. 127) | Reverse primer 3 (SEQ ID No. 128) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 4 | Forward primer 4 (SEQ ID No. 129) | Reverse primer 4 (SEQ ID No. 130) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 8 | Template 1 | Forward primer 1 (SEQ ID No. 131) | Reverse primer 1 (SEQ ID No. 132) |
| | pET-22b- | CCACCTGCCCGTTCctcgagCACC | GGTGTAGGTTGCCACggtgaacggg |
| | IsPETase-cSP | ACCACCACCACCACTG | ccggcgctg |
| | Template 2 | Forward primer 2 (SEQ ID No. 133) | Reverse primer 2 (SEQ ID No. 134) |
| | pET-22b- | ccggcccgttcaccGTGGCAACCTACA | AGATCGCCACCGCACCGAAGG |
| | LCC-cSP | CCGTTTCGC | TCAGCGAGGTGCCTGTGGG |
| | Template 3 | Forward primer 3 (SEQ ID No. 135) | Reverse primer 3 (SEQ ID No. 136) |
| | pET-22b- | CACCTCGCTGACCTTCGGTGCG | GCCGCCGCCCATCGAGTGGCCC |
| | TfCut2-cSP | GTGGCGATCTCCCCCGGC | ATGACCGCCAGTCGG |
| | Template 4 | Forward primer 4 (SEQ ID No. 137) | Reverse primer 4 (SEQ ID No. 138) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 5 | Forward primer 5 (SEQ ID No. 139) | Reverse primer 5 (SEQ ID No. 140) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 6 | Forward primer 6 (SEQ ID No. 141) | Reverse primer 6 (SEQ ID No. 142) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 7 | Forward primer 7 (SEQ ID No. 143) | Reverse primer 7 (SEQ ID No. 144) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 9 | Template 1 | Forward primer 1 (SEQ ID No. 145) | Reverse primer 1 (SEQ ID No. 146) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 147) | Reverse primer 2 (SEQ ID No. 148) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 149) | Reverse primer 3 (SEQ ID No. 150) |
| | pET-22b- | CACCTCGCTGACCTTCGGTGCG | GCCGCCGCCCATCGAGTGGCCC |
| | TfCut2-cSP | GTGGCGATCTCCCCCGGC | ATGACCGCCAGTCGG |
| | Template 4 | Forward primer 4 (SEQ ID No. 151) | Reverse primer 4 (SEQ ID No. 152) |
| | pET-22b- | GCGGTCATGGGCCACTCGATGG | cgttctcgcaggcgaagatcagCACCGGTA |
| | LCC-cSP | GCGGCGGCGGGAC | CCGACGTGTTGAACG |
| | Template 5 | Forward primer 5 (SEQ ID No. 153) | Reverse primer 5 (SEQ ID No. 154) |
| | pET-22b- | CGTTCAACACGTCGGTACCGGT | CTCCACGTACACCTTcgcattgcgcg |
| | IsPETase-cSP | Gctgatcttcgcctgcgagaacg | acatgctgtc |
| | Template 6 | Forward primer 6 (SEQ ID No. 155) | Reverse primer 6 (SEQ ID No. 156) |
| | pET-22b- | catgtcgcgcaatgcgAAGGTGTACGT | CAGAGGAACTGGGTGTAGCGG |
| | LCC-cSP | GGAGCTCGACAATG | GTGTCGTTATCCACCCACAGCT |
| | | | TC |
| 10 | Template 1 | Forward primer 1 (SEQ ID No. 157) | Reverse primer 1 (SEQ ID No. 158) |
| | pET-22b- | cgcgaactgcagcctcgagCACCACCAC | CCGCGAAACGGTGTAGGTTGCC |
| | TfCut2-cSP | CACCACCACTG | ACGGAGAAGGGGCCGCTG |
| | Template 2 | Forward primer 2 (SEQ ID No. 159) | Reverse primer 2 (SEQ ID No. 160) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 161) | Reverse primer 3 (SEQ ID No. 162) |
| | pET-22b- | CACCTCGCTGACCTTCGGTGCG | GCTCCACGTACACCTTGCTGAT |
| | TfCut2-cSP | GTGGCGATCTCCCCCGGC | GGAGCTCGGCAGG |
| | Template 4 | Forward primer 4 (SEQ ID No. 163) | Reverse primer 4 (SEQ ID No. 164) |
| | pET-22b- | CCGAGCTCCATCAGCAAGGTGT | ggtggagtagcgcgtGTCGTTATCCAC |
| | LCC-cSP | ACGTGGAGCTCGAC | CCACAGCTTC |
| | Template 5 | Forward primer 5 (SEQ ID No. 165) | Reverse primer 5 (SEQ ID No. 166) |
| | pET-22b- | GTGGGTGGATAACGACacgcgctac | CAGTGGTGGTGGTGGTGGTGctc |
| | IsPETase-cSP | tccaccttcg | gaggctgcagttcgcggtgcgg |
| 11 | Template 1 | Forward primer 1 (SEQ ID No. 167) | Reverse primer 1 (SEQ ID No. 168) |
| | pET-22b- | CCACCTGCCCGTTCctcgagCACC | GGGAGACGTTCTCCTCGCTGAC |
| | LCC-cSP | ACCACCACCACCACTG | CGAGAACGGCCCGTCGGCCGT |
| | | | G |
| | Template 2 | Forward primer 2 (SEQ ID No. 169) | Reverse primer 2 (SEQ ID No. 170) |
| | pET-22b- | CACGGCCGACGGGCCGTTCTCG | GCTCCACGTACACCTTGCTGAT |
| | TfCut2-cSP | GTCAGCGAGGAGAACGTCTCC | GGAGCTCGGCAGG |
| | | C | |
| | Template 3 | Forward primer 3 (SEQ ID No. 171) | Reverse primer 3 (SEQ ID No. 172) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 4 | Forward primer 4 (SEQ ID No. 173) | Reverse primer 4 (SEQ ID No. 174) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 12 | Template 1 | Forward primer 1 (SEQ ID No. 175) | Reverse primer 1 (SEQ ID No. 176) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 177) | Reverse primer 2 (SEQ ID No. 178) |
| | pET-22b- | CAGCGGCCCCTTCTCCGTGGCA | GATGGTGTCGATGGTGATGACG |
| | LCC-cSP | ACCTACACCGTTTCGC | ACGAAGCCGTGCGATGCCAAC |
| | | | CGC |
| | Template 3 | Forward primer 3 (SEQ ID No. 179) | Reverse primer 3 (SEQ ID No. 180) |
| | pET-22b- | CGGTTGGCATCGCACGGCTTCG | GCTCCACGTACACCTTGCTGAT |
| | TfCut2-cSP | TCGTCATCACCATCGACACCAT | GGAGCTCGGCAGG |
| | | C | |
| | Template 4 | Forward primer 4 (SEQ ID No. 181) | Reverse primer 4 (SEQ ID No. 1821) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 13 | Template 1 | Forward primer 1 (SEQ ID No. 183) | Reverse primer 1 (SEQ ID No. 184) |
| | pET-22b- | CCACCTGCCCGTTCctcgagCACC | AGATCGCCACCGCACCGAAGG |
| | LCC-cSP | ACCACCACCACCACTG | TCAGCGAGGTGCCTGTGGG |
| | Template 2 | Forward primer 2 (SEQ ID No. 185) | Reverse primer 2 (SEQ ID No. 186) |
| | pET-22b- | CACCTCGCTGACCTTCGGTGCG | GCCGCCGCCCATCGAGTGGCCC |
| | TfCut2-cSP | GTGGCGATCTCCCCCGGC | ATGACCGCCAGTCGG |
| | Template 3 | Forward primer 3 (SEQ ID No. 187) | Reverse primer 3 (SEQ ID No. 188) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 4 | Forward primer 4 (SEQ ID No. 189) | Reverse primer 4 (SEQ ID No. 190) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 5 | Forward primer 5 (SEQ ID No. 191) | Reverse primer 5 (SEQ ID No. 192) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 6 | Forward primer 6 (SEQ ID No. 193) | Reverse primer 6 (SEQ ID No. 194) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 14 | Template 1 | Forward primer 1 (SEQ ID No. 195) | Reverse primer 1 (SEQ ID No. 196) |
| | pET-22b- | GAAGCTGTGGGTGGATAACGA | GCCGCCGCCCATCGAGTGGCCC |
| | TfCut2-cSP | CACCCGCTACACCCAGTTCCTC | ATGACCGCCAGTCGG |
| | | TG | |
| | Template 2 | Forward primer 2 (SEQ ID No. 197) | Reverse primer 2 (SEQ ID No. 198) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 3 | Forward primer 3 (SEQ ID No. 199) | Reverse primer 3 (SEQ ID No. 200) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 4 | Forward primer 4 (SEQ ID No. 201) | Reverse primer 4 (SEQ ID No. 202) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 15 | Template 1 | Forward primer 1 (SEQ ID No. 203) | Reverse primer 1 (SEQ ID No. 204) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 205) | Reverse primer 2 (SEQ ID No. 206) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 207) | Reverse primer 3 (SEQ ID No. 208) |
| | pET-22b- | CACCTCGCTGACCTTCGGTGCG | cgcaggcgaagatcagCGTCGGCACGG |
| | TfCut2-cSP | GTGGCGATCTCCCCCGGC | TGACGCTGC |
| | Template 4 | Forward primer 4 (SEQ ID No. 209) | Reverse primer 4 (SEQ ID No. 210) |
| | pET-22b- | CGTCACCGTGCCGACGctgatcttcg | CTCCACGTACACCTTcgcattgcgcg |
| | IsPETase-cSP | cctgcgagaacg | acatgctgtc |
| | Template 5 | Forward primer 5 (SEQ ID No. 211) | Reverse primer 5 (SEQ ID No. 212) |
| | pET-22b- | catgtcgcgcaatgcgAAGGTGTACGT | CAGAGGAACTGGGTGTAGCGG |
| | LCC-cSP | GGAGCTCGACAATG | GTGTCGTTATCCACCCACAGCT |
| | | | TC |
| 16 | Template 1 | Forward primer 1 (SEQ ID No. 213) | Reverse primer 1 (SEQ ID No. 214) |
| | pET-22b- | CCACCTGCCCGTTCctcgagCACC | GGTGTAGGTTGCCACggtgaacggg |
| | IsPETase-cSP | ACCACCACCACCACTG | ccggcgctg |
| | Template 2 | Forward primer 2 (SEQ ID No. 215) | Reverse primer 2 (SEQ ID No. 216) |
| | pET-22b- | ccggcccgttcaccGTGGCAACCTACA | AGATCGCCACCGCACCGAAGG |
| | LCC-cSP | CCGTTTCGC | TCAGCGAGGTGCCTGTGGG |
| | Template 3 | Forward primer 3 (SEQ ID No. 217) | Reverse primer 3 (SEQ ID No. 218) |
| | pET-22b- | CACCTCGCTGACCTTCGGTGCG | GCTCCACGTACACCTTGCTGAT |
| | TfCut2-cSP | GTGGCGATCTCCCCCGGC | GGAGCTCGGCAGG |
| | Template 4 | Forward primer 4 (SEQ ID No. 219) | Reverse primer 4 (SEQ ID No. 220) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 5 | Forward primer 5 (SEQ ID No. 221) | Reverse primer 5 (SEQ ID No. 222) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 17 | Template 1 | Forward primer 1 (SEQ ID No. 223) | Reverse primer 1 (SEQ ID No. 224) |
| | pET-22b- | CCACCTGCCCGTTCctcgagCACC | AGATCGCCACCGCACCGAAGG |
| | LCC-cSP | ACCACCACCACCACTG | TCAGCGAGGTGCCTGTGGG |
| | Template 2 | Forward primer 2 (SEQ ID No. 225) | Reverse primer 2 (SEQ ID No. 226) |
| | pET-22b- | CACCTCGCTGACCTTCGGTGCG | GCTCCACGTACACCTTGCTGAT |
| | TfCut2-cSP | GTGGCGATCTCCCCCGGC | GGAGCTCGGCAGG |
| | Template 3 | Forward primer 3 (SEQ ID No. 227) | Reverse primer 3 (SEQ ID No. 228) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| | Template 4 | Forward primer 4 (SEQ ID No. 229) | Reverse primer 4 (SEQ ID No. 230) |
| | pET-22b- | GAAGCTGTGGGTGGATAACGA | GTGGTGGTGctcgagGAACGGGC |
| | TfCut2-cSP | CACCCGCTACACCCAGTTCCTC | AGGTGGAGCGG |
| | | TG | |
| 18 | Template 1 | Forward primer 1 (SEQ ID No. 231) | Reverse primer 1 (SEQ ID No. 232) |
| | pET-22b- | GAAGCTGTGGGTGGATAACGA | GCTCCACGTACACCTTGCTGAT |
| | TfCut2-cSP | CACCCGCTACACCCAGTTCCTC | GGAGCTCGGCAGG |
| | | TG | |
| | Template 2 | Forward primer 2 (SEQ ID No. 233) | Reverse primer 2 (SEQ ID No. 234) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 19 | Template 1 | Forward primer 1 (SEQ ID No. 235) | Reverse primer 1 (SEQ ID No. 236) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 237) | Reverse primer 2 (SEQ ID No. 238) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 239) | Reverse primer 3 (SEQ ID No. 240) |
| | pET-22b- | CACCTCGCTGACCTTCGGTGCG | GCCGCCGCCCATCGAGTGGCCC |
| | TfCut2-cSP | GTGGCGATCTCCCCCGGC | ATGACCGCCAGTCGG |
| | Template 4 | Forward primer 4 (SEQ ID No. 241) | Reverse primer 4 (SEQ ID No. 242) |
| | pET-22b- | GCGGTCATGGGCCACTCGATGG | CGAGGTCGGCCCCGATGATCA |
| | LCC-cSP | GCGGCGGCGGGAC | GCACCGGTACCGACGTGTTGAA |
| | | | CG |
| | Template 5 | Forward primer 5 (SEQ ID No. 243) | Reverse primer 5 (SEQ ID No. 244) |
| | pET-22b- | GTTCAACACGTCGGTACCGGTG | GCTCCACGTACACCTTGCTGAT |
| | TfCut2-cSP | CTGATCATCGGGGCCGACCTCG | GGAGCTCGGCAGG |
| | Template 6 | Forward primer 6 (SEQ ID No. 245) | Reverse primer 6 (SEQ ID No. 246) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |
| 20 | Template 1 | Forward primer 1 (SEQ ID No. 247) | Reverse primer 1 (SEQ ID No. 248) |
| | pET-22b- | GAAGCTGTGGGTGGATAACGA | CCGCGAAACGGTGTAGGTTGCC |
| | TfCut2-cSP | CACCCGCTACACCCAGTTCCTC | ACGGAGAAGGGGCCGCTG |
| | | TG | |
| | Template 2 | Forward primer 2 (SEQ ID No. 249) | Reverse primer 2 (SEQ ID No. 250) |
| | pET-22b- | CAGCGGCCCCTTCTCCGTGGCA | AGATCGCCACCGCACCGAAGG |
| | LCC-cSP | ACCTACACCGTTTCGC | TCAGCGAGGTGCCTGTGGG |
| | Template 3 | Forward primer 3 (SEQ ID No. 251) | Reverse primer 3 (SEQ ID No. 252) |
| | pET-22b- | CACCTCGCTGACCTTCGGTGCG | GCTCCACGTACACCTTGCTGAT |
| | TfCut2-cSP | GTGGCGATCTCCCCCGGC | GGAGCTCGGCAGG |
| | Template 4 | Forward primer 4 (SEQ ID No. 253) | Reverse primer 4 (SEQ ID No. 254) |
| | pET-22b- | CCGAGCTCCATCAGCAAGGTGT | CAGAGGAACTGGGTGTAGCGG |
| | LCC-cSP | ACGTGGAGCTCGAC | GTGTCGTTATCCACCCACAGCT |
| | | | TC |

### Example 23

The present example provides an engineered strain containing the recombinant plasmid of Example 22 and a construction method thereof. The construction method comprises:

The recombinant plasmid of Example 22 was transformed into a BL21(DE3) competent cell and cultured using an ampicillin-containing medium to obtain a positive recombinant, which was the engineered strain containing the recombinant plasmid of Example 22.

### Example 24

The present example provides a method for inducing expression the engineered strain of Example 23 and a method for purifying the target protein.

The engineered strain of Example 23 was inoculated into an LB liquid medium to culture overnight at 37° C., 220 r/min; the overnight culture solution was inoculated into a fresh LB liquid medium at an inoculation amount of 1% to culture at 37° C., 220 r/min to reach the OD600 about 0.8; IPTG with a volume percentage of 0.1% (v/v) was added to induce expression at 16° C. for 20 h to obtain a fermentation culture. The fermentation culture was centrifuged at 4000 rpm for 15 min, and the centrifuged engineered strain wet cells were collected.

The centrifuged engineered strain wet cells were resuspended in a disrupting buffer, and the resuspended engineered strain wet cells were subjected to a disrupting treatment using a high-pressure cell disruption instrument. The disrupted solution was centrifuged at 10000 rpm for 60 min to remove cell debris, and the supernatant was loaded into a Ni-NTA column to absorb the target protein. The non-specifically adsorbed impurity protein was cleaned by a washing buffer. The target protein was eluted using an elution buffer and the eluate was concentrated via a protein concentration tube.

The formulations of LB liquid medium were: 5 g/L of yeast extract, 10 g/L of tryptone, 10 g/L of sodium chloride.

The formulations of disrupting buffer were: 50 mM Tris-HCl, 150 mM NaCl, 10 mM Imidazole, pH=7.5.

The formulations of washing buffer were: 50 mM Tris-HCl, 150 mM NaCl, 20 mM Imidazole, pH=7.5.

The formulations of elution buffer were: 50 mM Tris-HCl, 300 mM NaCl, 300 mM Imidazole, pH=7.5.

The foregoing descriptions are only the preferred examples of the present application, but are not intended to limit the present application. Any modifications, equivalent substitutions, or improvements made within the spirit and principles of the present application shall fall within the scope of protection of the present application.

What is claimed is:

1. An IsPETase-8×Chimera recombinase, comprising an amino acid sequence modified from an amino acid sequence of a wild-type polyethylene terephthalate (PET) hydrolase by replacing at least one of the amino acid sequences set forth in SEQ ID NOs: 1 to 8, wherein the amino acid sequence of the wild-type PET hydrolase comprises the amino acid sequences set forth in SEQ ID NOs: 1 to 8; wherein
the amino acid sequence set forth in SEQ ID NO: 1 is replaced by the amino acid sequence set forth in SEQ ID NO: 9 or SEQ ID NO: 10;
the amino acid sequence set forth in SEQ ID NO: 2 is replaced by the amino acid sequence set forth in SEQ ID NO: 11 or SEQ ID NO: 12;
the amino acid sequence set forth in SEQ ID NO: 3 is replaced by the amino acid sequence set forth in SEQ ID NO: 13 or SEQ ID NO: 14;
the amino acid sequence set forth in SEQ ID NO: 4 is replaced by the amino acid sequence set forth in SEQ ID NO: 15;
the amino acid sequence set forth in SEQ ID NO: 5 is replaced by the amino acid sequence set forth in SEQ ID NO: 16 or SEQ ID NO: 17;
the amino acid sequence set forth in SEQ ID NO: 6 is replaced by the amino acid sequence set forth in SEQ ID NO: 18;
the amino acid sequence set forth in SEQ ID NO: 7 is replaced by the amino acid sequence set forth in SEQ ID NO: 19; and
the amino acid sequence set forth in SEQ ID NO: 8 is replaced by the amino acid sequence set forth in SEQ ID NO: 20.

2. The IsPETase-8×Chimera recombinase of claim 1, wherein the amino acid sequences set forth in SEQ ID NOs: 2, 3, 4, 5 and 7 are replaced.

3. The IsPETase-8×Chimera recombinase of claim 2, wherein the amino acid sequence set forth in SEQ ID NO: 3 is replaced by the amino acid sequence set forth in SEQ ID NO: 14.

4. The IsPETase-8×Chimera recombinase of claim 1, wherein the amino acid sequence set forth in SEQ ID NO: 3 is replaced by the amino acid sequence set forth in SEQ ID NO: 14.

5. The IsPETase-8×Chimera recombinase of claim 1, wherein the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 17, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

6. The IsPETase-8×Chimera recombinase of claim 1, wherein the amino acid sequence set forth in SEQ ID NO: 1 is replaced by the amino acid sequence set forth in SEQ ID NO: 10, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 17, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

7. The IsPETase-8×Chimera recombinase of claim 1, wherein the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

8. The IsPETase-8×Chimera recombinase of claim 1, wherein the amino acid sequence set forth in SEQ ID NO: 2 is replaced by the amino acid sequence set forth in SEQ ID NO: 12, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

9. The IsPETase-8×Chimera recombinase of claim 1, wherein the amino acid sequence set forth in SEQ ID NO: 1 is replaced by the amino acid sequence set forth in SEQ ID NO: 9, the amino acid sequence set forth in SEQ ID NO: 2 is replaced by the amino acid sequence set forth in SEQ ID NO: 11, the amino acid sequence set forth in SEQ ID NO: 3 is replaced by the amino acid sequence set forth in SEQ ID NO: 14, the amino acid sequence set forth in SEQ ID NO: 4 is replaced by the amino acid sequence set forth in SEQ ID NO: 15, the amino acid sequence set forth in SEQ ID NO: 5 is replaced by the amino acid sequence set forth in SEQ ID NO: 16, the amino acid sequence set forth in SEQ ID NO: 6 is replaced by the amino acid sequence set forth in SEQ ID NO: 18, the amino acid sequence set forth in SEQ ID NO: 7 is replaced by the amino acid sequence set forth in SEQ ID NO: 19, and the amino acid sequence set forth in SEQ ID NO: 8 is replaced by the amino acid sequence set forth in SEQ ID NO: 20.

10. A method for hydrolyzing PET using the IsPETase-8×Chimera recombinase of claim 1, comprising: adding PET to a glycine-sodium hydroxide (NaOH) buffer with a pH of 8.0 to 10.0 and a concentration of 40 millimoles per liter (mM) to 60 mM so that a final concentration of PET is 60 milligrams per milliliter (mg/mL) to 100 mg/mL; adding the IsPETase-8×Chimera recombinase to the solution so that a final concentration of the IsPETase-8×Chimera recombinase is 400 nanomoles per liter (nM) to 600 nM; and incubating the mixture for 1 to 3 days at a temperature of 30° C. to 50° C. and a rotation speed of 100 revolutions per minute (rpm) to 300 rpm.

11. A nucleic acid encoding the IsPETase-8×Chimera recombinase of claim 1.

12. A recombinant plasmid, comprising the nucleic acid of claim 11.

13. A method for constructing the recombinant plasmid of claim 12, comprising: using plasmid pET-22b-IsPETase, plasmid pET-22b-TfCut2 and plasmid pET-22b-LCC as templates, and performing PCR to obtain linear vector fragments corresponding to each template; mixing each of the linear vector fragments corresponding to each template with a ligase to form circular recombinant plasmids; transforming each of the circular recombinant plasmids into a T1 competent cell, and obtaining a recombinant plasmid pET-22b-IsPETase-cSP, a recombinant plasmid pET-22b-TfCut2-cSP and a recombinant plasmid pET-22b-LCC-cSP by ampicillin-containing medium screening and plasmid extraction; wherein a nucleotide sequence of an IsPETase-cSP gene in the recombinant plasmid pET-22b-IsPETase-cSP is set forth in SEQ ID NO: 51, and an amino acid sequence of an IsPETase-cSP enzyme is set forth in SEQ ID NO: 52; a nucleotide sequence of a TfCut2-cSP gene in the recombinant plasmid pET-22b-TfCut2-cSP is set forth in SEQ ID NO: 53, and an amino acid sequence of a TfCut2-cSP enzyme is set forth in SEQ ID NO: 54; a nucleotide sequence of a LCC-cSP gene in the recombinant plasmid pET-22b-LCC-cSP is set forth in SEQ ID NO: 55, and an amino acid sequence of a LCC-cSP enzyme is set forth in SEQ ID NO: 56;
using at least two of the recombinant plasmid pET-22b-IsPETase-cSP, the recombinant plasmid pET-22b-TfCut2-cSP and the recombinant plasmid pET-22b-LCC-cSP as templates to perform PCR separately to generate PCR amplified fragments, ligating the PCR amplified fragments to generate a ligation product, transforming the ligation product into a T1 competent cell, screening using the ampicillin-containing medium and performing plasmid extraction to obtain the recombinant plasmid.

14. An engineered strain, comprising the recombinant plasmid of claim 12.

15. A method for constructing an engineered strain, comprising: transforming the recombinant plasmid of claim 12 into a BL21(DE3) competent cell, and culturing the BL21(DE3) competent cell using an ampicillin-containing medium to obtain a positive recombinant.

16. A method for hydrolyzing PET using the engineered strain of claim 14, comprising: culturing the engineered strain in an LB liquid medium to reach OD600 ranging from 0.7 to 0.9, adding 0.05% to 0.2% of IPTG (isopropylthio-β-galactoside) to the medium, cooling to 14° C. to 20° C. to induce expression for 16 hours (h) to 24 h, centrifuging, and collecting wet cells of the engineered strain;
after breaking down the wet cells of the engineered strain, centrifuging to remove cell debris, eluting a centrifuged supernatant with an elution buffer as an elution solvent by using a Ni-NTA packing column, collecting an eluate, and concentrating the eluate to obtain a protein concentrate containing proteins;
adding the protein concentrate to a glycine-sodium hydroxide buffer with a pH of 8.0 to 10.0 and a concentration of 40 mM to 60 mM, so that the concentration of the proteins is 400 nM to 600 nM, and hydrolyzing PET at 30° C. to 60° C.; wherein a concentration of glycine in the glycine-sodium hydroxide buffer is 50 mM to 200 mM.
